(19) United States
(12) Patent Application Publication
(10) Pub. No.: US 2016/0089261 A1
(43) Pub. Date: Mar. 31, 2016

(54) Respiratory Treatment Devices for Airway Obstruction Therapy and the Like

(71) Applicant: Kenneth Quinn, Saskatoon (CA)

(72) Inventor: Kenneth Quinn, Saskatoon (CA)

(21) Appl. No.: 14/853,155

(22) Filed: Sep. 14, 2015

Related U.S. Application Data
(60) Provisional application No. 62/056,833, filed on Sep. 29, 2014.
(60) Provisional application No. 62/075,997, filed on Nov. 6, 2014.

(51) Int. Cl. A61F 5/56; A61M 16/08; A61M 16/06; A61M 16/00

(52) U.S. Cl. CPC A61F 5/56; A61M 16/0057; A61M 16/0875; A61M 16/0683; A61M 16/0605; A61M 16/0666

(57) ABSTRACT

Unique cervical collars and patient interfaces for sleep apnea therapy. One cervical collar features a chin-supporting member whose topside is movable relative to a chest-abutting member. The top side is biased upwardly against the underside of the wearer's chin to maintain an erect head position and closed lower jaw position when the user is asleep, while allowing intentional opening of the mouth during consciousness. The chest-abutting member features a support by which a patient interface is carried thereon to avoid or reduce the need for a separate head-worn securing means. A patient interface employs a venturi-like airway configuration to create a vacuum for securing the patient interface to facial or nasal tissue without head straps or the like. Electrical contacts on the cervical collar detect a positional status of the wearer's lower jaw, and are used to control an operational state of a pressurized air source coupled to the interface.

### CROSS REFERENCE TO RELATED APPLICATIONS

This application claims benefit under 35 U.S.C. 119(e) of Provisional Application Ser. No. 62/056,833, filed Sep. 29, 2014; and Provisional Application Ser. No. 62/075,997, filed Nov. 6, 2014, the entireties of which are incorporated herein by reference.

### FIELD OF THE INVENTION

The present invention relates generally to devices for treating respiratory conditions, and more particularly to unique cervical collars, patient interfaces and combinations thereof for use in treating sleep apnea or other airway disturbances.

### BACKGROUND

The use of a cervical collar to maintain a wearer's head in an erected or rearwardly inclined position and/or maintain the lower jaw in a raised position closing the wearer's mouth has previously proposed as a method of treating sleep apnea. U.S. Patent Application Publication US 2012/0234330 is an example of one such disclosure, in which the user's chin rests atop a cervical collar braced against the wearer's chest. U.S. Pat. No. 7,789,843 discloses another neck secured apparatus supporting the chin upwardly away from the wearer's chest for the treatment of sleep apnea, and includes an adjustable jack for adjusting the fit of the apparatus to suit a particular user.

U.S. Pat. No. 5,289,829 is another example of a neck-worn apparatus acting between the wearer's chest and chin for maintaining a closed position of the wearer's mouth, but is prescribed for the purpose of preventing snoring. U.S. Patent Application Publication 2012/0047656 discloses a spring-loaded apparatus also worn between the chin and chest area to maintain a desired position of the wearer's head. U.S. Pat. No. 5,904,662 discloses a cervical collar incorporating an inflatable bladder to adjust the vertical dimension and deflection resistance of the collar.

Another treatment for sleep apnea is the use of a CPAP (continuous positive airway pressure) machine for non-intrusive ventilation of a sleep apnea sufferer. The machine provides a continuous pressurized stream of air to the user's airways through the nasal passages via a respiratory patient interface, which is typically a face-worn mask overlying the full nasal area of the user in sealed contact with the wearer's facial tissue, a nasal pillow held upwardly against the user's nares in sealed condition therewith to convey air directly into the nasal passages, or nasal prongs that carried reach upwardly into the nasal passages and radially expand into contact with the walls of the nasal passages to achieve a sealed state of the airway to maintain the positive pressure applied by the CPAP machine.

Examples of prior respiratory patient interfaces for nasal-based ventilation include those shown in U.S. Patent Application Publications 2011/0067704, which includes clips for securing the interface to the wearer's septum; 2012/0152255, which includes optional use of two blowers for independent pressure control to each nasal passage; 2008/0216835, which incorporates a built in CPAP machine with an audible alarm function; and 2010/0113957, which includes means for testing or monitoring of a sleep apnea condition; and U.S. Pat. No. 8,573,219, which includes use of a radially expandable cushion or a vacuum to hold nasal prongs in place.

Other references with potential relevancy to the present application include U.S. Pat. Nos. 3,964,474; 5,123,425; 6,935,335; 7,789,837; 8,382,692; 8,578,938; 5,989,193; 2,580,628; 3,640,270; 4,556,065; 7,054,677; 2,284,058; 5,507,718; 7,040,581; and 7,182,082; and U.S. Patent Application Publications 2005/0183721; 2005/0190065; and 2014/0123977.

Applicant has designed new cervical collars, patient interfaces and combinations thereof that provide improved or alternate solutions in the area of sleep apnea therapy.

### SUMMARY OF THE INVENTION

According to a first aspect of the invention, there is provided an apparatus for treatment of sleep apnea or other respiratory disturbances, the apparatus comprising:

a cervical collar comprising a chin-supporting member arranged to be worn anteriorly of the user's neck in a position receiving the user's chin on a top side of the chin-supporting member;

a support feature on the cervical collar at a position residing anteriorly of the user's neck when worn, the support feature being arranged to carry a respiratory patient interface on the cervical collar.

Preferably the cervical collar further comprises a stationary chest-abutting member arranged to be worn anteriorly of the user's neck in a position against the user's chest, and the top side of the chin-supporting member is movable downwardly relative to the chest-abutting member under to accommodate opening of the user's jaw while in a conscious state, the top-side of the chin-supporting member being biased upwardly to force closing of the user's jaw while in an unconscious state, and the support feature being disposed on the stationary chest-abutting member at a position situated anteriorly of the chin-supporting member.

According to a second aspect of the invention, there is provided an apparatus for treatment of sleep apnea or other respiratory disturbances, the apparatus comprising:

a cervical collar comprising:
a stationary chest-abutting member arranged to be worn anteriorly of the user's neck in a position against the user's chest,
a chin-supporting member arranged to be worn anteriorly of the user's neck in a position receiving the user's chin on a top side of the chin-supporting member, the top side of the chin-supporting member being movable downwardly relative to the chest-abutting member under to accommodate opening of the user's jaw while in a conscious state, and being biased upwardly to force closing of the user's jaw while in an unconscious state; and

a support feature on the stationary chest-abutting member arranged to carry a respiratory patient interface thereon.

Preferably the support feature is configured to engage an air conveyance conduit to which the respiratory patient interface is coupled to receive supply air from said air conveyance conduit.

Preferably the support feature is arranged to hold the ventilator airway conduit in a position reaching superiorly from the second member to convey air to a nasal respiratory patient interface.

The support feature may comprise a force application member arranged to urge the respiratory patient interface into a working position engaged to a body of the user.

The respiratory patient interface may comprise an air delivery mask, in which case the force application member is preferably arranged to urge the air delivery mask posteriorly of the user for facial engagement with the body of the user.

The force application member may be a spring lever standing upwardly from the support feature at a front end thereof with a distal end of the spring lever biased rearwardly to urge the air delivery mask posteriorly of the user.

Alternatively, the patient interface may comprises nasal pillows, in which case the force application member is preferably arranged to urge the air nasal pillows superiorly of the user for nasal engagement with the body of the user.

In such instance, preferably the support feature comprises a stationary support component held stationary to the second member and a movable support component movably coupled to the stationary support component and biased upwardly relative thereto, the movable support component being arranged to couple to the nasal pillows.

In such instance, preferably the movable support component is arranged to couple to the nasal pillows via an air conveyance conduit that is coupled to said nasal pillows to deliver air thereto.

The support feature may comprise an adjustment mechanism for adjusting a position of the respiratory patient interface without disconnection thereof from the support feature.

Preferably the support feature comprises a stationary component coupled to the outer member and a movable component threadingly mated to the stationary component and displaceable relative to the stationary component by relative rotation between the threadingly mated components.

Preferably the threadingly mated components are arranged for relative upward and downward displacement to adjust the position of the respiratory patient interface in a superior-inferior direction of the user.

The cervical collar may comprise an inflatable chamber connected or connectable to a pressurized air supply to enable inflation of the chamber to varying levels to a modify a height at which the support feature is located in order to adjust a position at which the respiratory patient interface is carried.

In one embodiment, the support comprises a first attachment point for connection of a respiratory patient interface thereto, and the cervical collar comprises a second attachment point for connection of an air conveyance conduit thereto, and an internal air path extending between the first and second attachment points within the cervical collar to transfer air from the air conveyance conduit to the respiratory patient interface.

In one embodiment, the chin-supporting member and the chest-abutting member carry respective electrical contacts thereon that are positioned to come into and out of contact with one another under relative movement between the top side of the chin-supporting member and the chest-abutting member, thereby detecting changes in a positional status of the chin received on the top side of the inner member.

In such instance, preferably the respective electrical contacts are positioned to come into and out of contact with one another under relative movement between the top side of the inner member and the chest-abutting member in a superior-anterior direction of the user, thereby detecting changes in an open/closed state of the user's jaw.

In such instance, preferably the electrical contacts are positioned to come into contact under inferior urging of the top side of the chin-supporting member toward the chest-abutting member by a jaw-opening action of the user.

Preferably the contacts are connected to a control circuit of a respiratory ventilation machine to alter an output thereof in response to a change in a state of contact between the electric contacts.

Preferably the electrical contacts and the control circuit of the respiratory ventilation machine are arranged to reduce an output air pressure of the respiratory ventilation machine under inferior urging of the top side of the inner member toward the outer member by a jaw-opening action of the user.

The cervical collar may comprise a first attachment point defined at the support for connection of a respiratory patient interface thereto, second attachment point for connection of an air conveyance conduit thereto, and an internal air path extending between the first and second attachment points within the cervical collar to transfer air from the air conveyance conduit to the respiratory patient interface.

According to a third aspect of the invention, there is provided a cervical collar for positioning a lower jaw of a user in treatment of sleep apnea or other respiratory disturbances, the cervical collar comprising:

an inner member arranged to be worn anteriorly of the user's neck in a position receiving the chin of the user on a top side of the inner member with a rear side of the facing posteriorly of the user, a front side of the inner member facing anteriorly of the user, and two lateral sides facing laterally of the user;

an outer member spanning across the front side of the inner member from one end of the outer member; and

a securing band attached to the ends of the outer member for passing posteriorly around the neck of the user to secure the inner and outer members in place;

wherein the top side of the inner member is movable relative to the outer member to accommodate movement of the chin in at least superior and inferior directions relative to the chest on which the outer member resides during use while supporting the chin of the user from below.

Preferably the front side of the inner member is convexly curved and a rear side of the inner member facing toward the front side of the inner member is concavely curved to accommodate the inner member in a void between the ends of the outer member.

Preferably the inner member is segment-shaped or crescent shaped, and the outer member is arc-shaped or crescent-shaped.

Preferably the inner member is biased in a superior direction of the user in order to urge the chin in a jaw-closing direction.

The outer member may comprise a first attachment point for connection of a respiratory patient interface thereto, second attachment point for connection of an air conveyance conduit thereto, and an internal air path extending between the first and second attachment points within the outer member to transfer air from the air conveyance conduit to the respiratory patient interface.

According to a third aspect of the invention, there is provided a cervical collar for positioning a lower jaw of a user in treatment of sleep apnea or other respiratory disturbances, the cervical collar comprising:

a first member arranged to be worn anteriorly of the user's neck in a position receiving the user's chin on a top side of the first member;

a second member arranged to be worn anteriorly of the user's neck in a position residing against the user's chest; and

a securing band attached to the second member for passing posteriorly around the neck of the user to secure the inner and outer members in place;

wherein at least the top side of the first member is movable relative to the second member to accommodate movement of the chin in at least superior and inferior directions relative to the chest on which the second member resides during use while supporting the chin of the user from below; and

wherein the first and second members carry respective electrical contacts thereon that are positioned to come into and out of contact with one another under relative movement between the top side of the first member and the second member, thereby detecting changes in a positional status of the chin received on the top side of the first member.

According to a fifth aspect of the invention, there is provided a respiratory patient interface comprising:

a primary air inlet for receiving incoming air from an air conveyance conduit connected or connectable to the respiratory patient interface;

an air outlet from which outgoing air is provided to a user,

an air passage connected between the primary air inlet and the air outlet to guide airflow therebetween; and

at least one secondary inlet joined with the air passage at an intermediate location between the primary air inlet and the air outlet;

wherein the air passage constricts in a direction moving from the primary air inlet to where the second inlet joins with the air passage to induce a venturi effect creating a vacuum at the secondary inlet, which is located at a position on the respiratory patient interface that resides adjacent a tissue area of the user so that the vacuum at the secondary inlet holds the air deliver device to said tissue area.

In one embodiment, the respiratory patient interface is a nasal pillow, and the at least one secondary inlet resides at an outer periphery of said nasal pillow to hold the nasal pillow to interior nasal tissue of the user.

In another embodiment, the respiratory patient interface is an air mask for wearing over a facial area of the user, and the at least one secondary inlet resides at a facial contact rim of the air mask to hold the air mask to facial tissue of the user.

According to a sixth aspect of the invention, there is provided a respiratory patient interface comprising:

a pair of nasal pillows joined together by a connection spanning therebetween;

a pneumatically controlled septum holding device attached to the connection for use in securing the nasal pillows in a working position received in or communicating with nasal passages of the user by gripping a nasal septum that separates said nasal passages, wherein the pneumatically controlled septum holding device comprises first and second parts for respective receipt within said nasal passages, and at least one of the first and second parts of said septum holding device being movable between a normal default position and an actuated position by supply of pressurized air to said at least one of said parts.

Preferably said at least one of the first and second parts of said septum holding device is a hollow expandable member having an inlet through which the pressurized air is received to expand said hollow expandable member into the actuated position, in which said hollow expandable member expands toward the other of said at least one of the first and second parts to grip the nasal September between said first and second parts.

Preferably both of the first and second parts are movable between said normal default position and said actuated position.

Preferably both of the first and second parts are hollow expandable members.

### BRIEF DESCRIPTION OF THE DRAWINGS

One embodiment of the invention will now be described in conjunction with the accompanying drawings in which:

FIG. 1 is a schematic side view of a first embodiment sleep apnea treatment collar with an upper chin-supporting member spring biased upwardly from a chest-abutting lower member to urge the wearer's lower jaw upwardly into a closed position.

FIG. 2 is a side view of a second embodiment sleep apnea treatment collar featuring a singular spring-like member to similarly force the wearer's lower jaw upwardly away from the chest into the closed position.

FIG. 3 is a side view of a third embodiment sleep apnea treatment collar having a support feature thereon for carrying a respiratory patient interface in a working position thereon.

FIG. 4 is a side view of a first variant of the third embodiment sleep apnea treatment collar of FIG. 3, in which the collar is of the type shown in FIG. 1 and the respiratory patient interface is a pair of nasal pillows biased upwardly into a working position in contact with the wearer's nares.

FIG. 5 is a side view of a second variant of the third embodiment sleep apnea treatment collar, in which the sleep apnea collar incorporates an inflatable internal bladder, the expansion of which increases the vertical height of the collar to hold the lower jaw closed and force the nasal pillows upwardly into the working position.

FIG. 6 is a side view of a third variant of the third embodiment sleep apnea treatment collar, in which the respiratory patient interface is a facial mask and the support feature includes a spring lever urging the facial mask rearward into contact with the wearer's facial tissue.

FIG. 7 is a side view of a fourth variant of the third embodiment sleep apnea treatment collar, in which the support feature has threadingly mated upper and lower components for movable support of the respiratory patient interface to adjust a height of the working position.

FIG. 8 is a side view of a fifth variant of the third embodiment sleep apnea treatment collar, in which in which the support feature has a spring-loaded movable upper support component for biasing the nasal pillows upwardly into the working position.

FIG. 9 is a is a side view of a sixth variant of the third embodiment sleep apnea treatment collar, in which the a spring function is provided by the support feature or air hose to hold the nasal pillows in place.

FIG. 10 is a side view of a seventh variant of the third embodiment sleep apnea treatment collar, in which the respiratory patient interface connects to the apnea treatment collar separately from an air conveyance conduit through which air is conveyed from a CPAP machine.

FIG. 11 is a side view of an eighth variant of the third embodiment sleep apnea treatment collar featuring an EEG sensor connected to the support feature in order to detect a sleep status of the wearer for use thereof in control of the CPAP machine.

FIG. 12A is a rear view of a fourth embodiment respiratory patient interface in the form of a facial mask using a venturi-shaped air path to create suction at a series of ports in a contact rim of the mask for vacuum-based retention of the mask in sealed relation to the wearer's facial tissue.

FIG. 12B is a schematic elevational view of a variant of the fourth embodiment respiratory patient interface, in which the venturi-shaped air path is used in a nasal pillow for vacuum based retention of the pillow to the nasal wall of the user.

FIG. 13 is a schematic elevational view of a fifth embodiment respiratory patient interface featuring a pneumatically actuated nasal septum clip.

FIG. 14 is a side view of a sixth embodiment bed-mounted support for an air conveyance conduit and respiratory patient interface of a CPAP system.

FIG. 15A a median cross-sectional view of a seventh embodiment sleep apnea treatment collar featuring inner and outer segments, of which the inner segment receives the wearer's chin and is movable relative to the outer segment.

FIG. 15B is an overhead plan view of the sleep apnea treatment collar of FIG. 15A.

FIG. 15C is a median cross-sectional view of a first variant of the seventh embodiment sleep apnea treatment collar.

FIG. 15D is an overhead plan view of the sleep apnea treatment collar of FIG. 15C.

FIG. 16A is a side view of a second variant of the seventh embodiment sleep apnea treatment collar featuring cooperating electrical contacts on the inner and outer segments that come into and out of contact with one another under relative movement of the inner and outer segments to detect open and closed states of the wearer's mouth and make use of the detected states in automated control of a CPAP machine.

FIG. 16B is a schematic illustration of how the contacts of the sleep apnea treatment collar of FIG. 16A form a switch in a control circuit of a CPAP machine.

FIG. 17 is a side view of a third variant of the seventh embodiment sleep apnea treatment collar, which adds a support feature of the type shown in FIG. 3 to the outer segment of the collar shown in FIG. 7.

FIG. 18 is a side view of an eight embodiment CPAP system featuring two separate air conveyance conduits coupled to two respective outputs of a CPAP machine for independent pressure control of the two nasal passages of the user.

FIG. 19 is a schematic elevational view of a ninth embodiment respiratory patient interface featuring a pair of nasal pillows, of which only one pillow accommodates through-flow of a pressurized air stream from the CPAP machine, while the other pillow uses said pressurized air stream to dilate into a radially expanded state engaged with the nasal walls of the user to retain the pillows in place.

FIG. 20 is a schematic elevational view of a variant of the ninth embodiment respiratory patient interface in which the expandable nasal pillow uses separate mechanical means for dilation thereof instead of the pressurized air supply from the CPAP machine.

In the drawings like characters of reference indicate corresponding parts in the different figures.

### DETAILED DESCRIPTION

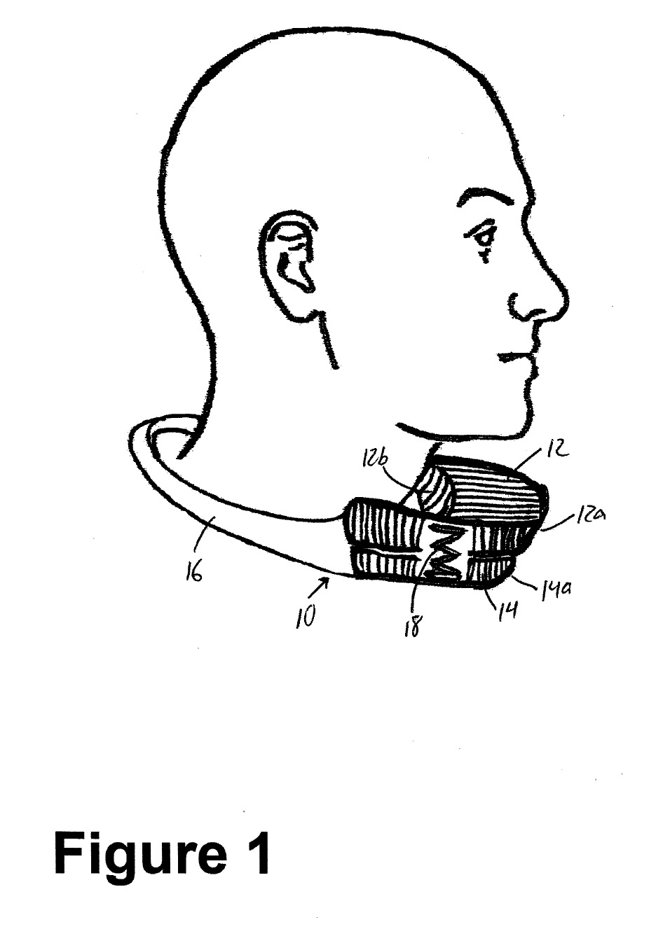

FIG. 1 shows a cervical collar device 10 having an upper segment 12 and a lower segment 14, wherein the upper segment is suitable for the positioning and weight support of the wearer's chin on a top side of the upper segment at a position forwardly (i.e. anteriorly) of the neck, and the lower segment is suitable for positioning its underside on the upper chest area of the wearer when the segments are retained in position by a neck band or strap 16 spanning between the ends of the two segments behind the neck of the wearer. The upper segment is connected to the lower segment by a coil-type compression spring 18 that urges the upper segment upwardly away from the lower segment and against the underside of the wearer's chin. This creates a spring action such that a user is able to move their head up or down or open their mouth, with the spring action as a counter force against such motion. When the user is asleep and their muscles relaxed, the spring action is sufficient to keep the user's head properly positioned and maintain the user's mouth in a closed position due to the upward force on the user's lower jaw from beneath the chin. The spring action may be adjustable to be suitable for all sizes and weights of male and female users.

The upper and lower segments in the drawing each comprise a substantially solid body of foam or other material, with a blind hole recessed into the underside of the upper segment and a matching blind hole recessed into the topside of the lower segment, whereby the two recesses cooperate to substantially enclose the spring. While the term segment is used herein refer to each of these upper and lower members, 12, 14, it is not intended to denote a particular shape (e.g. a circular segment), and the members may have various shapes. In the illustrated embodiment, the body of each member is crescent shaped, where a front side 12a, 14a of each body facing forwardly (i.e. anteriorly) away from the wearer's neck is convexly curved about an axis lying longitudinally (i.e. in the superior-inferior direction) of the wearer's body, and a rear side of each body (the upper one of which can be seen at 12b) faces rearwardly (i.e. posteriorly) and concavely toward the wearer's neck. The band 16 connects to the ends of the upper and lower segments, which reside laterally beside the neck in positions facing laterally outward therefrom. The body of each segment is solid in front of and behind the spring-accommodating bores or recesses so as to enclose the spring mechanism within the cervical device. The device and segments of the device may be covered with suitable materials for comfort, ventilation and appearance.

In the first illustrated embodiment, where both the upper and lower segments are each joined to a respective end of the band or strap 16 that prevents or limits relative movement between the segments at this common connection, the relative movement between the two segments is generally pivotal in nature. While the illustrated embodiment employs a coil-type compression spring, other spring types may be employed, including gas springs, plate springs, torsion springs, etc.

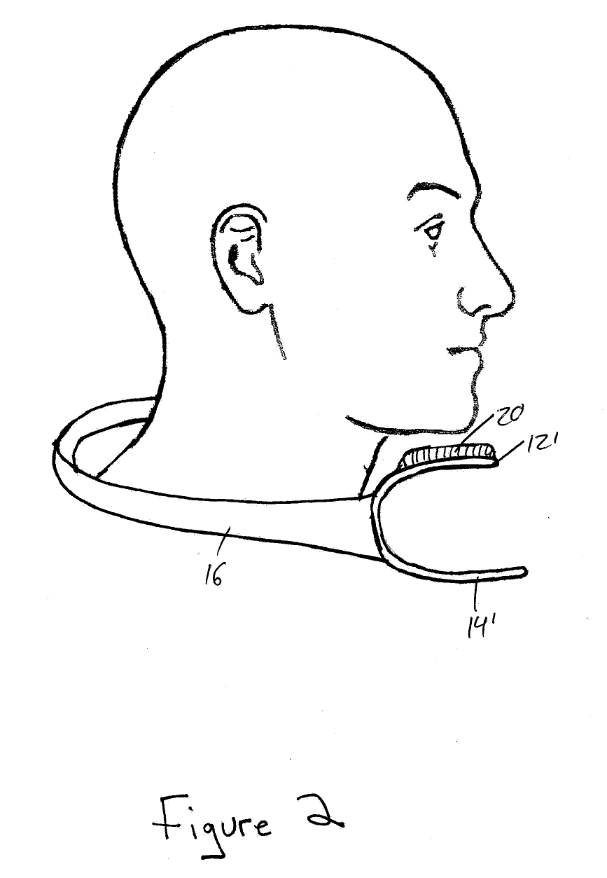

Turning to FIG. 2, in another embodiment the upper and lower segments 12′, 14′ may be of a wire or tubing design rather than solid bodies of foam or other material, and for example may have a piece of padding 20 mounted atop the upper segment for comfort against the wearer's chin. The device may be constructed of a singular wire or tubing piece, whereby the upper and lower segments are integrally formed by the shape and content of the tubing or wire, and the springing action of the segments away from one another may be accomplished by the design and structural content of said device. That is, the upper and lower segments may be biased away from one another by a resiliency of the wire or tubing structure itself, which tends to force the segments a default distance apart that is sufficient to keep the segments in respective contact with the chin and chest area of the user. Alternatively, a separate spring, such as the compression spring of FIG. 1, may have its opposing ends attached to the upper and lower wire or tubing segments to bias them apart and resist deflection thereof toward one another in supplement to any such resistance provided by the overall wire or tubing structure.

The forgoing devices may also be designed to provide independent horizontal movement of the upper segment from the lower segment while maintaining the spring action. In the first embodiment, wherein the upper and lower segments are separate members, or in instances of the second embodiment where the upper and lower segments are not integral with one another, this may be accomplished with a variety of rail, groove, bearing or track assemblies. In instances of the second embodiment where the upper and lower segments are integral with one another, flexing of the structure in directions other than a generally pivotal movement about a laterally lying-axis may accommodate such multi-directional movement.

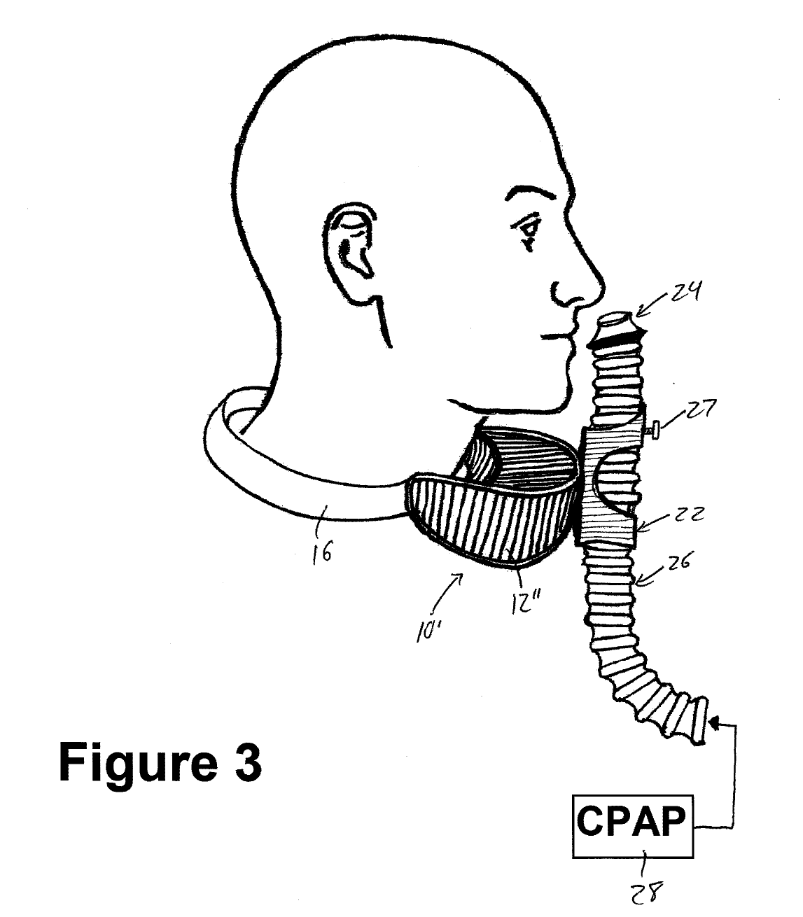

FIG. 3 illustrates a cervical collar 10′ with means to accommodate a respiratory patient interface 24. In this instance, the collar is a singular crescent-shaped body 12″ residing anteriorly of the neck with the aforementioned crescent shape having a concave front side facing forwardly/anteriorly of the neck and an opposing concave rear side facing rearwardly/posteriorly toward the neck. In this instance, the body is formed of a resiliently compressible material (e.g. foam) so that the resilience of the material urges the chin-supporting top side of the body upwardly away from the chest-resting underside of the collar that rests on the wearer's chest in order to maintain an erect or rearwardly inclined position of the head and urge the lower jaw into the closed position. A structural support feature 22 is affixed to the front side 12a of the body 12″. The structural support 22 may be composed of plastic or similar substance that provides a rigid support to accommodate a respiratory patient interface 24, such as a face-engaging CPAP mask and/or nare-engaging nasal pillows, and may include support and accommodation for a CPAP hose 26 that defines an air conveyance conduit for conveying a pressurized air stream from an source (e.g. the airflow generator of a CPAP machine 28) to the respiratory patient interface 24.

In the illustrated embodiment, the support 22 is has a through-bore that lies in the longitudinal (superior-inferior) direction of the wearer and is of suitable diameter to accommodate the CPAP hose 26 therein, whereby the hose 26 passes longitudinally through the support 22 and upwardly (superiorly) therefrom toward the wearer's nose such that the patient interface 24 carried at the upper end of the hose 26 is suitably positioned to supply air to the nasal passages of the wearer. In a conventional manner, the hose 26 may be a corrugated plastic hose. The hose 26 can be pulled in either direction through the bore of the support to adjust the hose's longitudinal position 26 relative to the support, and thereby likewise adjust the longitudinal position of the patient interface 24 in order to ensure a proper working position thereof in relation to the wearer's nose. One the desired position is obtained, a holder 27 in the form of a set screw, clamp or other suitable securing mechanism is engaged with the support 22 and the hose 26 in order to the hose in the attained position. The figure shows a set-screw holder, wherein the threaded shaft of the set screw is engaged in a threaded radial hole in the circumferential wall of the support 22 such that manual rotation of the head of the set screw in opposing directions tightens and loosens the shaft of the set screw against the hose 26 to secure and release the same against the opposing side of the support's circumferential wall. Having means to accommodate the support of the patient interface and/or CPAP hose, the cervical collar provides the user with a strapless, more comfortable support of the CPAP therapy components.

It will be understood that the invention disclosed herein, in various embodiments thereof, although making reference to a CPAP device and CPAP therapy, should not be deemed exclusive to that therapy but understood to include various other methods of treating sleep apnea, which may include biPAP devices, vPAP devices and devices that only deliver humidified air. The air flow described also may be any suitable deliverable gas. The devices may also be employed for respiratory conditions other than sleep apnea that may benefit from the features of the devices and methods disclosed herein.

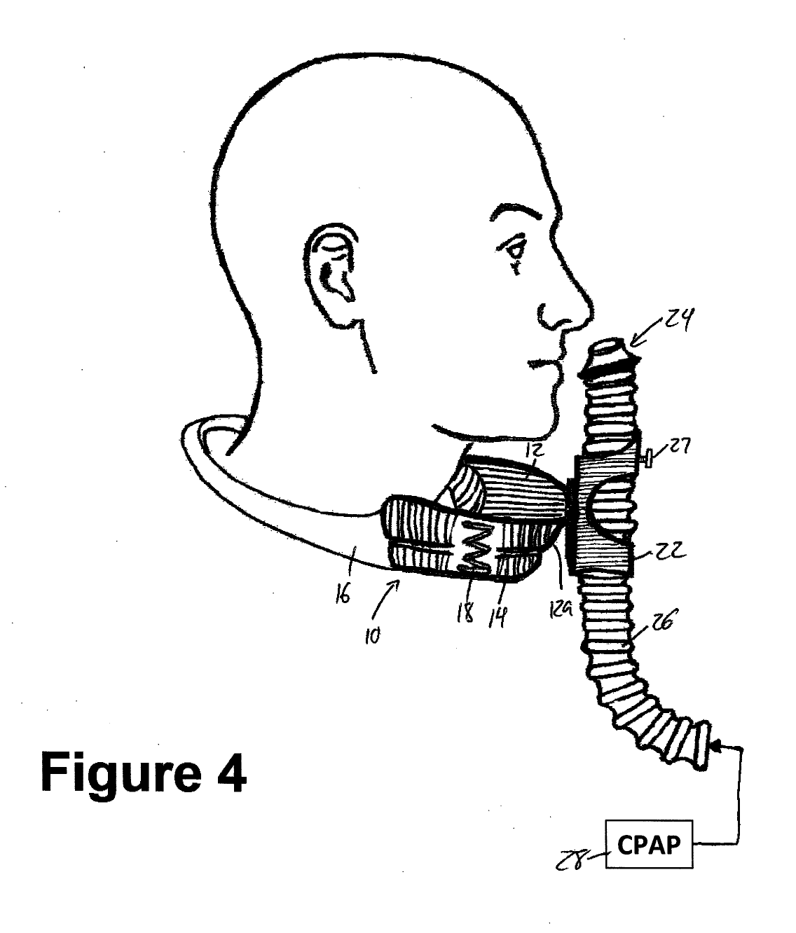

Turning to FIG. 4, the support 22 of FIG. 3 may likewise be employed on a cervical device 10 like that of FIG. 1, with a spring action forcing the upper member 12 away from the lower member 14. The spring action may be accomplished with a variety of springing mechanisms such as coil/helical, tension, extension, compression, torsion, constant, variable or gas spring types. The shape of the spring may be coil, flat, wire, tubular or machined. The spring action is sufficient to keep the users head properly positioned and the user's mouth closed when the user is asleep. As an alternative to the use of a separate spring, the spring action may be provided by air pressure from a CPAP device 28 such that when the user is awake and the CPAP pressure low or off, the user is able to move their head, open their mouth or talk normally, but once asleep and the CPAP air pressure increases, the user's mouth is kept closed by the CPAP air flow. The spring mechanism may be mechanical and aided by the air flow or the cervical device may include an interior bladder or foam filled bladder design such that when the air pressure increases the bladder expands upward becoming more rigid as the pressure increases.

The support 22 may be attached to the upper, lower or both segments of the cervical collar. FIG. 4 shows the support attached to the front side 12a of the upper segment 12, but in other embodiments, the support 22 may be attached solely to the lower segment 14 at an area thereof protruding forwardly (anteriorly) out from under the upper segment, such that any unlikely opening of the wearer's lower jaw with sufficient force to overcome spring bias of the upper segment 12 of the cervical device 10 doesn't act to pull the patient interface 24 downwardly. In such cases, the mounting of the support 22 to the lower segment that rests in a stationary position on the user's chest isolates the support from any movement experienced by the chin-supporting upper segment 12. On the other hand, attachment of the support 22 to the spring biased upper segment of the cervical may present some benefit in use of the spring force to help maintain an optimal position of the patient interface when the user's mouth is closed. The support bracket 22 may be flexible or removably mountable to the cervical device in different selectable positions to allow the user to better position the CPAP mask or nasal pillows.

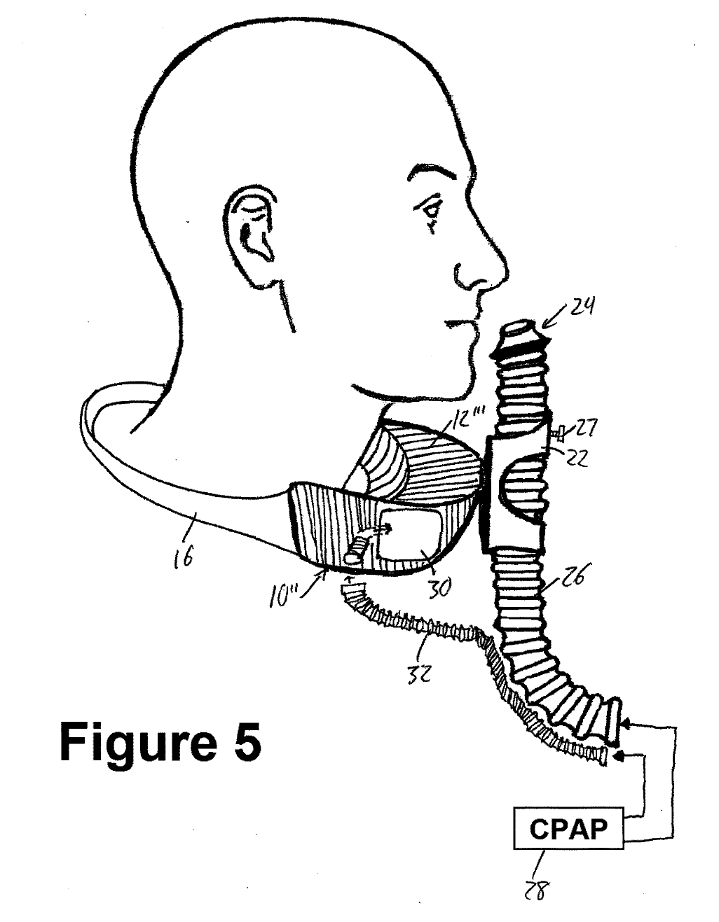

Turning to FIG. 5, a cervical collar 10″ comprised of a neck band or strap 16 carrying a singular crescent-shaped body of foam 12′″ or similar material has at least one air tight bladder or foam filled bladder 30 embedded therein. The body is 12′″ is placed under the chin and on top of the upper chest, and again has a support 22 to accommodate a CPAP mask, nasal pillows or other patient interface and/or a CPAP hose. The shape and material of the cervical collar is able to maintain the user's mouth in a closed position by receiving air flow in the bladder 30, for example received from a CPAP device 28 such as that from which air is provided to the patient interface 24. This causes the bladder or bladders 30 to expand and become more rigid. The size and expansion of the collar may be adjusted by various means of constricting or expanding the outer collar that houses the bladder, or regulating the air pressure of the bladder 30. It should be understood that the expanding cervical collar housing the bladder may function in a manner that solely keeps the users mouth closed by receiving air from the CPAP air flow, or may additionally or alternatively feature the described support means 22 to support and accommodate CPAP masks, nasal pillows, other patient interfaces and/or a CPAP hose, as well as all other embodiments as disclosed further in this patent.

With the expandable bladder 30 defining an inflatable chamber of the cervical collar 10″, the expansion of the bladder expands the height or thickness of the cervical collar (i.e. the dimension thereof lying longitudinally of the wearer), thus forcing the top side of the collar 10″ upwardly against the wearer's chin such that the user's mouth remains closed and the user's head remains positioned to allow free air flow without obstruction in the air passage. It should be understood that the air flow and resulting pressure in the bladder may originate from an alternate air flow device other than a CPAP device. The airflow device may be manually operated, may be attached to the cervical collar, may be electrically operated as a standalone device with a hose 32 providing the airflow to the bladder similar to the CPAP device. An electric airflow device feeding the bladder may be mains powered or battery operated, and may be enclosed in the cervical collar, or otherwise incorporated thereon. It should also be understood that this disclosed bladder of the invention may function with other embodiments as disclosed elsewhere in this patent, such as the humidified air flow described elsewhere herein. In one embodiment sensors provide data that determines when the user has fallen asleep, the cervical collar automatically inflates and remains inflated at an optimum pressure until the sensor data indicates the user is awake causing the bladder to deflate and allow the user to move their head and jaw freely.

With continued reference to FIG. 5, it will be appreciated that if the support 22 is attached to the cervical collar 10″ near the chin-contacting top side thereof, then the expansion of the bladder forces the top side of the cervical device 10″ further upward, and this expansion of the cervical device also moves the support 20 upward, and therefore may be used to likewise move the patient interface 24 upwardly. Accordingly, the level of inflation of the bladder can be used not only to control positioning of the head and lower jaw, but also to adjust the position of the patient interface and attached hose 26.

A cervical collar may have means to accommodate several types of patient interfaces 24, including full face masks, nose mask and nasal pillows, which may involve the inclusion of a force application member to urge the patient interface into a suitable working position.

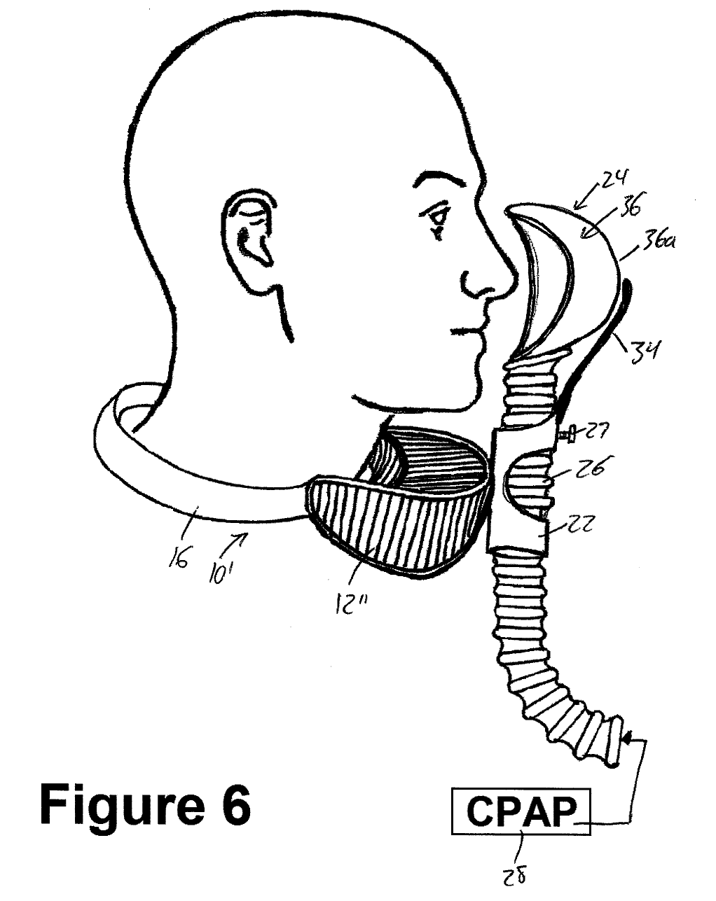

Turning to FIG. 6, a cervical collar designed for use with patient interfaces of the type featuring full face masks or nose type masks may such have a force application member in the form of a spring lever 34 standing upwardly from the support 22 at a front end thereof to abut against a closed front end 36a of the mask 36 that is carried at the top end of the hose 26. The lever 34 urges the mask 36 rearwardly (posteriorly) toward the wearer's face in a working position against the wearer's facial tissue around the nasal area thereof. As shown, the spring lever 34 may have a curved configuration better conforming to the closed front end of the mask 36. This application of force from the front of the mask differs from the conventional rearward pulling of the mask by straps extending posteriorly around the wearer's head, and accordingly may improve the comfort of the wearer by avoiding conventional mask straps. While FIG. 6 shows the lever-equipped support 22 on a single piece cervical collar 10′, it will be appreciated that it may be used on the other multi-piece collars described herein.

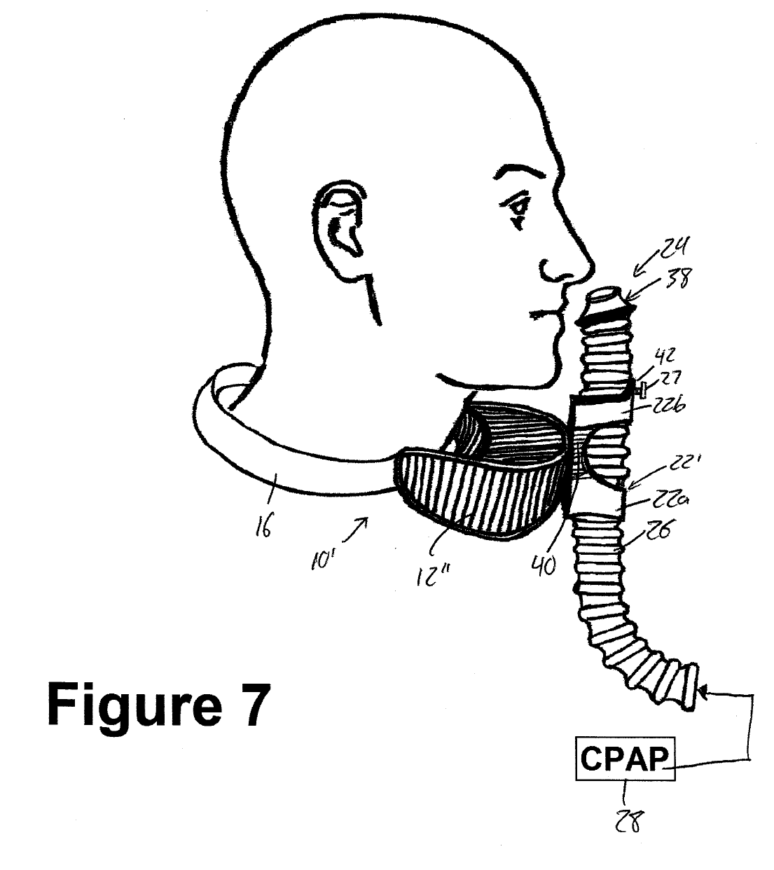

Turning to FIG. 7, one cervical collar for use with nasal pillows 38 employs a two-piece version of the support 22′, which features a first lower support component 22a attached to the cervical collar and having a longitudinal through bore to accommodate the CPAP hose, and a second upper support component 22b adjustably mounted to the lower component 22a to allow adjustment of a position of the upper support component 22b relative to the lower support component 22a in the longitudinal direction. In the illustrated embodiment, the upper support component 22b takes the form of an internally threaded collar whose internal threads are mated with corresponding external threads 40 of the lower support component 22a, whereby adjustment of the upper support component's position relative to the lower support component is accomplished by rotation of the upper support component relative to the lower support component about the longitudinal axis shared by the aligned bores of the two components. Such relative rotation longitudinally displaces the collar-shaped upper support component 22b along the lower support component. A raised portion 42 of the upper support component 22b, for example a portion of the collar wall of greater axial length than a remainder of the collar wall, is elevated above the threaded interface between the two components to provide an area at which a set screw, clamp or other holder 27 engages the CPAP hose or nasal pillows in order to secure the nasal pillows 38 to the adjustable upper support component 22b of the support 22′. Accordingly, raising and lowering of the upper support component 22b relative to the lower support component 22a raises and lowers the nasal pillows 38, whereby the nasal pillows can be adjusted in position without disconnection from the support 22′ in order to achieve the best fit of the nasal pillows with the nares of the wearer. The threaded engagement between the two components of the two-piece support thus provides an adjustment mechanism for adjusting a position of the respiratory patient interface 24 without disconnection thereof from the support feature. This allows the user to set the most comfortable position of the nasal pillows by simply loosening the holder 27, sliding the hose 26 upwardly or downwardly through the support 22 to achieve the desired position of the nasal pillows and then re-tightening the holder 27. The two piece support 22′ may similarly be used with a mask-type patient interface to allow adjustment of the mask position in the longitudinal direction by likewise rotating the upper support in clockwise and counterclockwise directions relative the lower support component. It will be appreciated that the upper support component may be affixed to the cervical collar, in which the case the CPAP hose would be held by the lower support component, which would be rotatably coupled to the upper component for longitudinal displacement relative thereto.

Turning to FIG. 8, there is shown another variant of the support-equipped cervical collar with a multi-piece support 22″ usable with nasal pillow patient interfaces 38. In the illustrated version, the lower support component 22c is of a shape that allows the upper support components 22d to be received within the lower support component such that a spring 44 in the lower support component 22c applies an upward longitudinal force to the bottom of the upper support component 22d, thereby allowing the upper support component 22d that carries the nasal pillows 38 to move independently of the lower support component 22c in the longitudinal direction. This allows the user to move their head up and down while maintaining the optimum upward force and longitudinal distance for the nasal pillows to provide a proper fit in the nasal cavities. The upper component is telescopically received within the lower support for longitudinal sliding thereto. Inside the lower component, the spring 44 acts against the lower end of the upper support component to urge the same longitudinally upward through the top end of the lower support, where the CPAP hose 26 or patient interface 24 is held to the upper support component by a set screw, clamp or other holder 27. The nasal pillows 38 of the patient interface 24 are thereby biased upwardly (superiorly) to ensure secure engagement with the nares of the wearer. The spring 44 may be removable such that the user is able to choose from springs of varying length and spring force.

In another embodiment (not shown), a non-adjustable single piece support 22 like those of FIG. 3 to 6, or the collar-attached support section of a multi-piece support like those of FIGS. 7 and 8, attaches to the cervical collar via a ball joint or other movable connection, for example to provide a swivel action allowing full directional movement of the support thus allowing a further range of motion of the structure while maintaining a proper CPAP mask or nasal pillow fit. In another embodiment, a multi-piece support may have a swivel-enabling connection (e.g. ball joint) between its upper and lower components, or at an attachment of one of said components to the cervical collar, to allow full directional movement between the collar-attached component and the interface-carrying component.

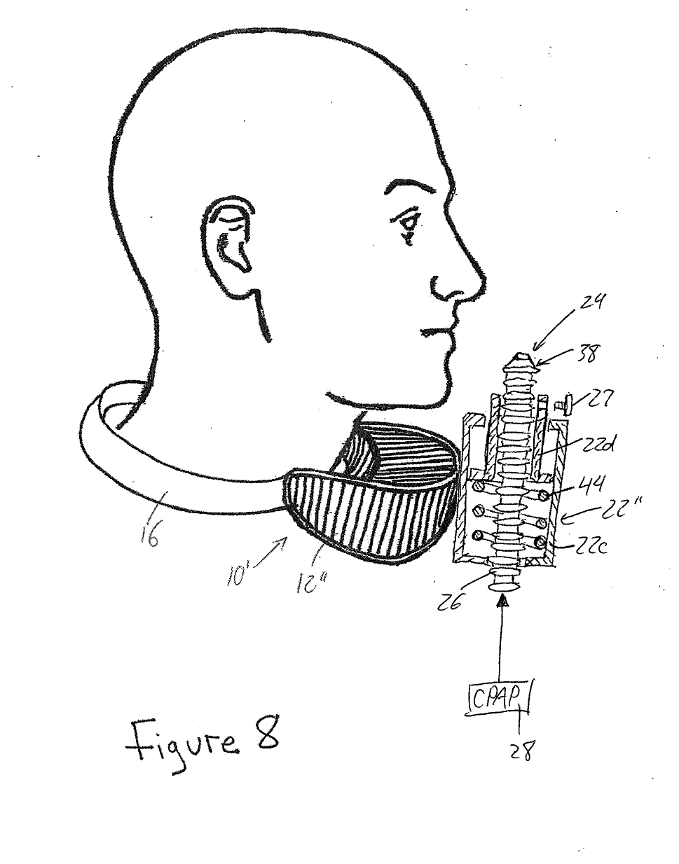

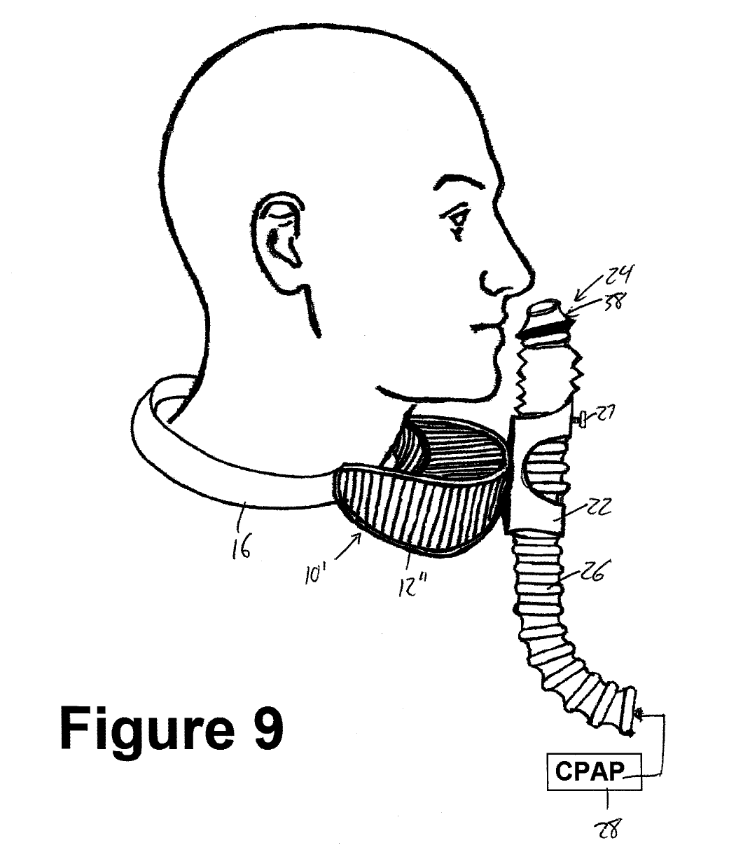

Turning to FIG. 9, the upper support includes a spring mechanism 45 on which the nasal pillows are attached so that the spring mechanism 45 forces the nasal pillows upwardly toward the nose of the user for consistent engagement with the user's nares in a working position. The spring can deflect in any direction to accommodate movement of the user's head while maintaining the working position of the nasal pillows. As shown, the spring mechanism may be a corrugated extension of the CPAP hose 26 that can be resiliently compressed in the axial direction, and resiliently deflect from a normally linear extending upright position into a tilted orientation in any direction. Alternatively, a compression spring mounted atop the support may be coiled around the CPAP hose 26 and abut against an underside of the nasal pillows. In another embodiment, a compression spring may be incorporated into the wall or interior space of the CPAP hose and urge the nasal pillows upwardly away from the point at which the hose 26 is secured to the support 22 by holder 27.

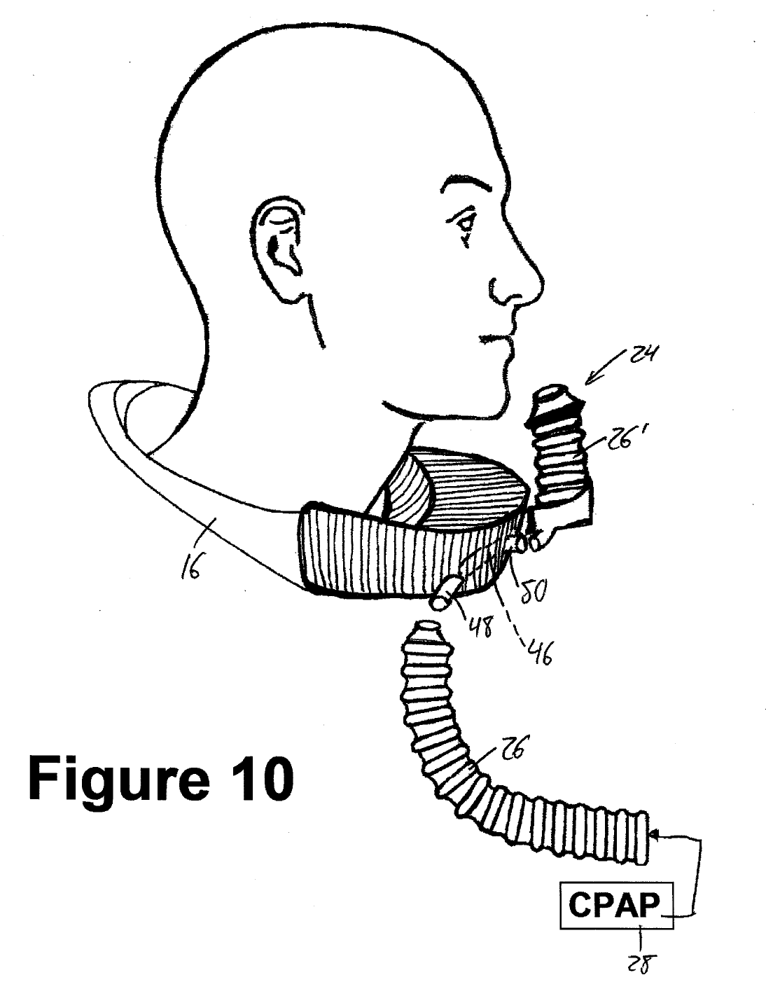

Turning to FIG. 10, the illustrated cervical collar has means for receiving the CPAP hose and patient interface 24 (e.g. CPAP mask or nasal pillows) independently of one another, allowing the CPAP mask or nasal pillow to move and function independently of the CPAP hose. That is, the patient interface 24 can be left in place in the working position on the collar, even if the CPAP hose 26 is disconnected. The cervical collar allows continuous airflow through at least one interior chamber or tube 46 that has independent connecting means for the CPAP mask or nasal pillow and CPAP hose, thus allowing better positioning of the CPAP hose for the user. That is, the collar has a first attachment point 48 at one end of the air path defined by the interior chamber or tube 46 for connection of the CPAP hose 26, and a second attachment point 50 at the other end of the air path for connection to the patient interface 24, for example by way of a shorter second hose 26′. This allows for the user to easily detach the main hose 26 from the collar such that the user does not have to remove the collar or interface 24 if they desire to get up and move around after they have prepared themselves for sleep.

In another embodiment (not shown) the at least one air chambers in the cervical collar allows for divergent airflow such that air flow is diverted to provide air flow and thus a cooling effect for the user in and around the neck area which may become heated by the users body temperature, the air flow may be regulated by a air flow regulating device presented to the user.

In another embodiment (not shown) the at least one air chamber in the cervical collar allows for divergent airflow such that air pressure from the CPAP machine provides tension to the hose and/or mask supports such that if the pressure of the air stops the masks and nasal pillows lose the tension needed to provide an airtight fit allowing the user to breathe properly in the case of a power or CPAP machine failure.

In another embodiment (not shown) the cervical collar has means to direct an air flow in a manner that makes it available to the user directly, the user may not have a CPAP device or mask and only require extra humidity which may be provided by a humidifier that adds the optimum humidity to an air flow through a hose which is received by the cervical collar. The hose may attach directly to the cervical collar and present the air to the user or attach to the cervical collar which directs the air to the user. This avoids the need to humidify a whole room in order for the user to breathe humidified air while they sleep since the air is introduced in proximity to the users mouth and nostrils.

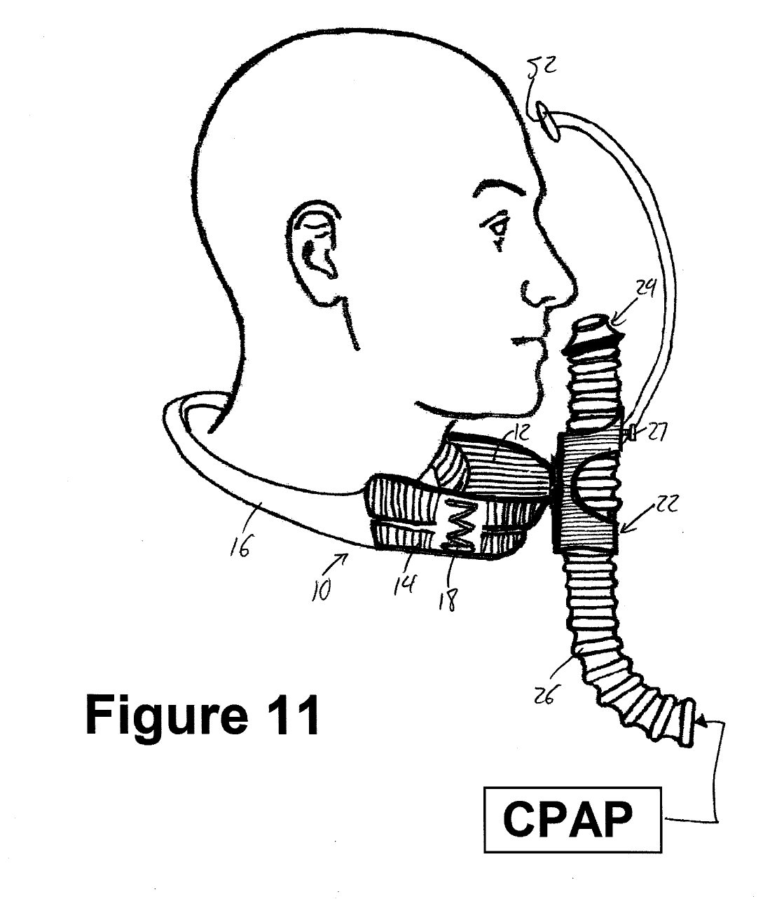

Turning to FIG. 11, the collar 10 and support 22 of FIG. 4 are additionally equipped with means to provide at least one attachable EEG sensor 52 to monitor a user's brain waves such that the resulting sensor output can be used by the CPAP machine to monitor the users sleep patterns such as REM sleep. The measurable data from the sensor may be used by the CPAP device in a control circuit thereof to turn the air supply function of the CPAP device 28 on when the user is asleep and off when the user is awake. The at least one attachable sensor 52 may be incorporated into the patient interface 24 or into the support structure 22, as illustrated, such that no further separate means of attachment of the sensor is necessary.

FIG. 12 illustrates use of the air flow from the CPAP machine 28 to cause a venturi effect in the patient interface 24, which is used to create a suction force for securing the patient interface in place, whether to supplement or fully replace the need for other securing means such as head straps or harnesses.

FIG. 12A shows incorporation of such a feature into a mask-type user interface 36′ worn on the face of the user in a position covering the nose thereof by way of mating with the surrounding facial tissue of the wearer at a facial contact rim 54 of the mask 36′. A base of the mask 36′ features a primary air inlet 56 to which the CPAP hose 26 is coupled at a lower end of the base, and an air outlet 58 opening upwardly from a topside of the base into an interior space 60 of the mask such that a pressurized air stream 62 from the CPAP machine 28 is delivered to this interior space 60. An air passage 64 joining the primary air inlet 56 to the air outlet 58 tapers toward the air outlet 58 to form a constriction 66 between the primary air inlet and the air outlet. A plurality of secondary inlets 68 are provided in the contact rim 54 of the mask at discrete positions spaced therearound, and are fluidly connected to the air passage 64 at the constriction 66 by one or more channels or conduits attached to or defined within the contact rim 54. By way of the venturi effect, the flow of the pressurized air stream 62 from the CPAP machine 28 through the constricted air passage creates suction at the secondary inlets 68, such that when the contact rim 54 of the mask is placed into contact with the facial tissue of the user to enclose the user's nose within the interior space 60 of the mask 36′, this suction secures the mask to the wearer's facial tissue. In a variant of the venturi-secured mask, the air flow from the CPAP machine is diverted through a separate enclosed channel in the CPAP mask that causes air flow to produce a venturi effect causing suction on the users skin thereby holding the CPAP mask in place, the air flow exhausting into the normal air flow of the CPAP mask and/or CPAP hose.

FIG. 12B shows incorporation of a similar venturi-based retention mechanism into a nasal pillow patient interface 38′. A base 69 of the nasal pillow 38′ features a primary air inlet 56′ reaching downwardly therefrom for coupling to the CPAP hose 26. A tapered nasal-penetrating body 70 of the nasal pillow 38′ sits atop the base and fits upwardly into the wearer's nasal passage when the base 69 is abutted against the boundaries of the user's nares in the worn position of the nasal pillow. The nasal-penetrating body features an internal conduit 72 with an air outlet 58′ that discharges air upwardly into the nasal passage through the narrower upper end 70a of the nasal-penetrating body to introduce the pressurized air stream 62 to the nasal passage. An air passage 64′ joining the primary air inlet 56 to the internal conduit 72 of the nasal-penetrating body 70 tapers toward the smaller-diameter internal conduit 72 from the larger diameter primary inlet 56, whereby the internal conduit 72 forms a constriction in the airflow path of the pressurized air stream from the CPAP machine 28. A plurality of secondary inlets 68′ are provided in the outer circumference of the nasal-penetrating body 70 and are in fluid communication with the constriction-defining internal conduit 72 via small branch tubes or tunnels 74 extending outward therefrom. By way of the venturi effect, the flow of the pressurized air stream 62 from the CPAP machine 28 through the constricted air flow path creates suction at the secondary inlets 68′, such that when the nasal penetrating body is received in the user's nasal passage, suction at the secondary inlets 68′ secures the nasal penetrating body to the surrounding nasal wall, thereby holding the nasal pillow 38′ in place. The venturi branches 74 may be arranged in a manner that causes the suction to be applied to the sides of the nasal septum in the nostrils, thereby holding the CPAP mask or nasal pillows in place. Alternatively, the nasal pillows are designed with a venturi tunnel in the lower nasal cavities such that suction is created on the outer side of the nasal cavity interior. This prevents over pressure of the nostrils which may causes distortion of the nostrils, and keeps the nasal pillows positioned. While FIG. 12B shows only one pillow, it will be appreciated that the patient interface may feature two pillows, one for each nasal passage, which may share a same CPAP hose from which two constricted flow paths diverge, or which may employ two respective CPAP hoses, one feeding each nasal pillow.

In another variant (not illustrated) of the venturi-secured patient interface, the air flow from the CPAP machine is directed to the interface 24 by a secondary hose, which may be attached to the primary hose, such that after the secondary air flow causes the venturi effect and resulting suction on the skin thereby holding the mask in place the air flow exhausting through the secondary hose which returns to the CPAP machine and away from the user.

In another embodiment (not illustrated), the venturi effect described above may be incorporated into the cervical collar disclosed above such that the venturi effect and resulting vacuum is in the area of the chin, the venturi tunnel incorporated in the cervical collar structural CPAP hose support.

In another embodiment (not illustrated) of the venturi effect as described above the exhausted air is directed through a cervical collar having air exhaust ports near the skin of the users neck and chin causing a cooling effect to enhance the comfort of the CPAP therapy, the cervical collar may also function as already described.

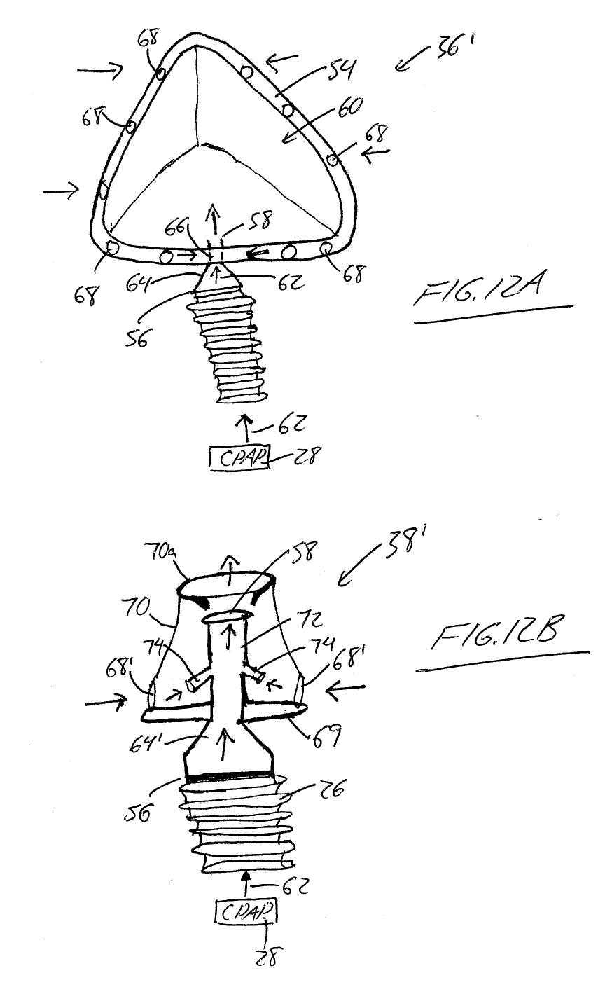

Turning to FIG. 13, one embodiment of a nasal pillow patient interface 38″ features a pair of nasal pillows 76 each having a base 69 and a tapered nasal-penetrating body 70 standing upwardly therefrom. A connection channel 78 joins the bases 69 of the two nasal pillows 76 together below the bases thereof and features an inlet port 80 for coupling to the CPAP hose 26 to distribute pressurized air to each nasal pillow through the channel 78. Between the two nasal pillows, there is a nasal septum holding device 80 that is attachable to the user's nasal septum that separates the nasal passages in order to hold the nasal pillows in place during use of the patient interface. In the illustrated embodiment, the septum holding device 80 is a pneumatically operated clip formed such that when positioned over the nasal septum, pneumatically supplied pressure is applied to both sides of the nasal septum in the nostrils to clamp the patient interface in place. To accomplish this, the clip features two hollow inflatable members 82, 84 symmetrically residing on opposite sides of a central plane cutting through the interface between the two nasal pillows. Each member has a proximal end 82a, 84a in fluid communication with the hollow interior of the connection channel 78 so as to receive pressurized air from the incoming supply of air from the CPAP machine 28. In an inflated state, the two inflatable members converge toward one another at distal ends 82b, 84b thereof situated opposite the proximal ends, whereby the inflated members abut against sides of the wearer's septum. The inflated state of each member imparts an increased rigidity thereto relative to the uninflated state of the member, whereby this stiffened state maintains the member in its septum gripping condition. Accordingly, the nasal-penetrating bodies 70 of the nasal pillows are inserted into the nasal passages to achieve a properly seated position with the larger-diameter bases 69 of the pillows abutted up against the boundaries of the user's nares, at which point the CPAP device 28 is activated to supply air to the patient interface, whereupon the inflatable members 80a, 80b of the septum holding device 80 are actuated from their normal default uninflated positions to their inflated positions forced against opposing sides of the septum to hold the patient interface in place. While the illustrated design uses the same primary stream 62 of pressurized air from the CPAP machine 28 that feeds the nasal pillows to activate the septum holding device 80, other embodiments may employ a separate secondary air source, for example provided by a secondary outlet of the CPAP machine.

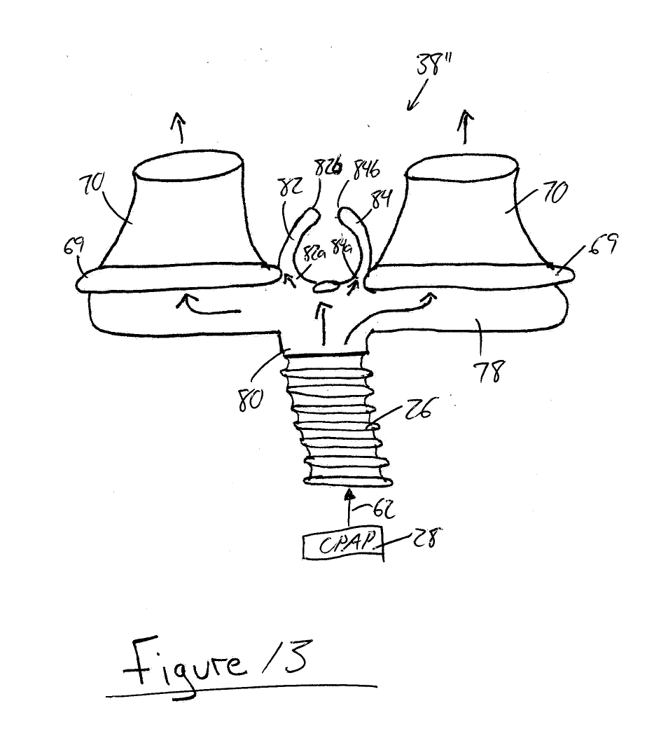

FIG. 14 shows a structural support with means to attach to a wall, ceiling, bed 86, headboard 88 or other structure, and which can accommodate a CPAP machine and/or a CPAP hose and interface assembly 24, 26 such that the CPAP user is able to have easy access to the CPAP equipment which is available in an overhead manner such that the CPAP hose 26 is not in contact with the user or the bedding surface. The structural support may include an automatic retractable system and/or a swivel system that keeps the CPAP hose and interface assembly at the proper positioning for the user while in use. The structural support may also include a receiving section for the patient interface 24 (whether a CPAP mask or nasal pillows) that provides a sterile environment for the assembly to be stored hygienically.

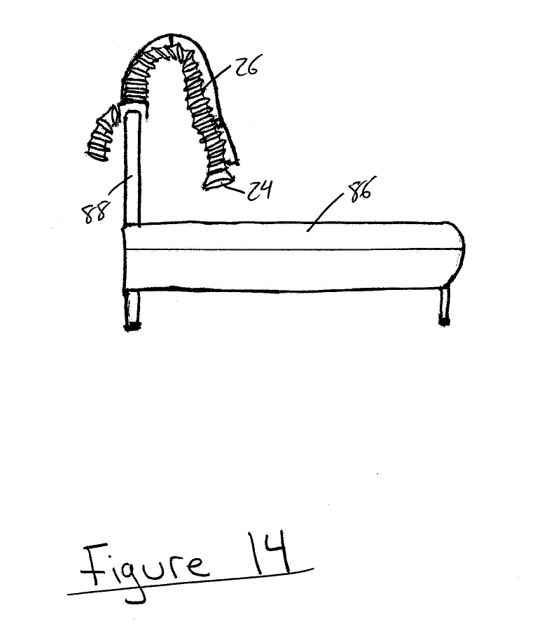

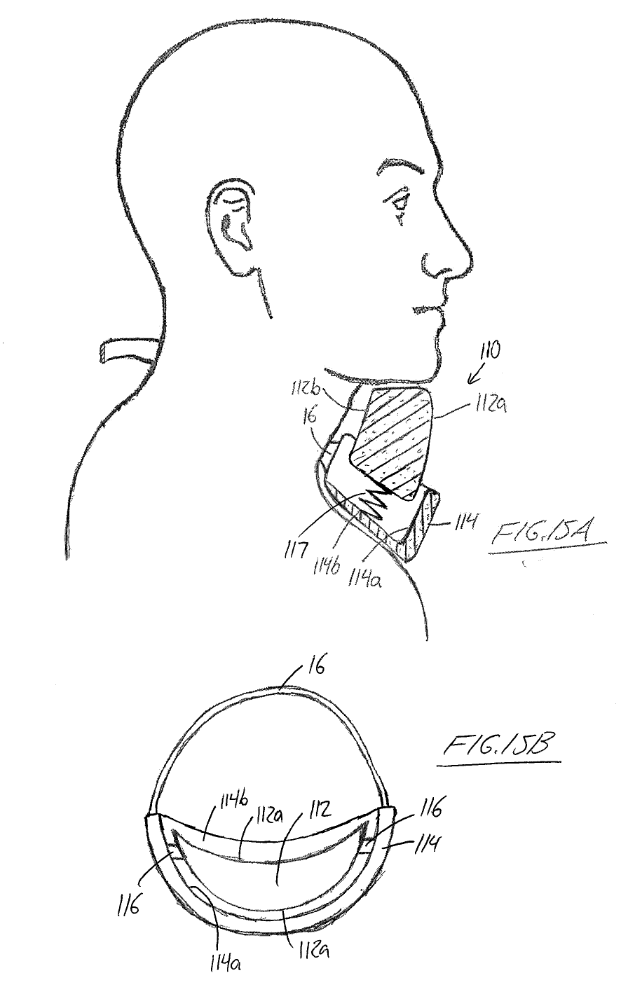

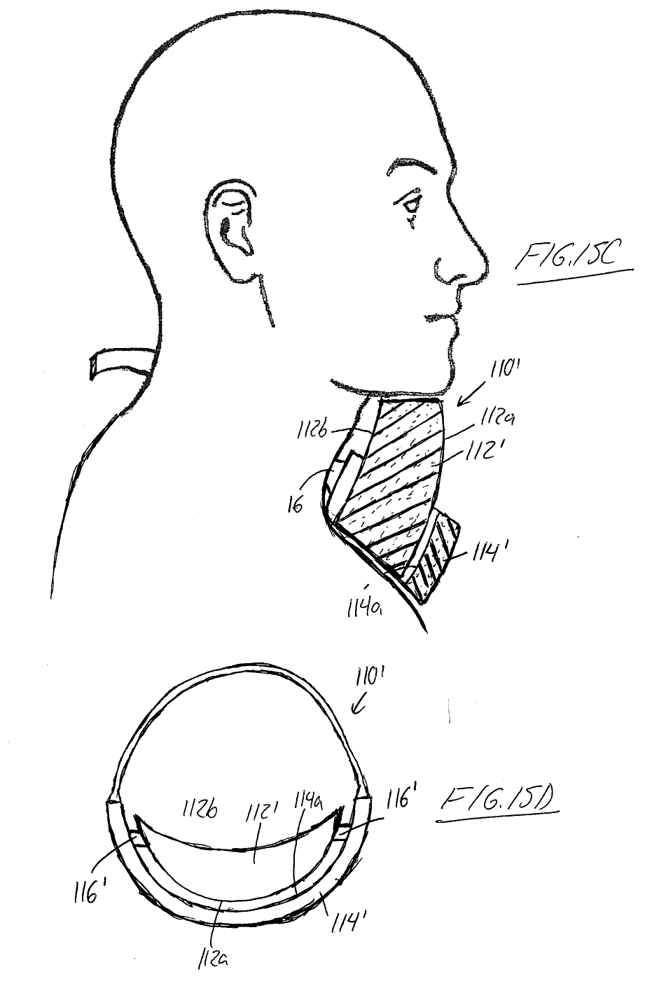

Turning to FIG. 15A, there is illustrated another two-segment cervical collar 110 in which an inner segment 112 residing nearer the wearer's neck than a cooperating outer segment 114 spanning across the front of the inner segment. The inner segment 112 is crescent shaped like the upper segment of the first embodiment of FIG. 1. Accordingly, a front side 112a of the upper segment faces forwardly (i.e. anteriorly) away from the wearer's neck and is convexly curved about an axis lying longitudinally (i.e. in the superior-inferior direction) of the wearer's body, and a rear side 112b of the inner segment faces rearwardly (i.e. posteriorly) and concavely toward the wearer's neck. The illustrated outer segment 114 is arc-shaped, with a concave rear side 114a of the arc facing the front side 112a of the inner segment and following therealong in a generally conforming manner with a slightly greater radius of curvature so as not to contact the inner segment. The arc-shaped outer segment 114 spans the full length of the convex front side 112a of the inner segment, which joins up to the concave rear side 112b thereof at two lateral sides of the inner segment that faces laterally outward from the wearer. Accordingly, the outer segment 114 fully surrounds the inner segment on the front and lateral sides thereof to substantially enclose the inner segment and thereby retain the same within a void space that is bound between the ends of the outer segment and between the neck of the wearer and the rear side of the outer segment. A neck band or strap 16 connects to the two ends of the arc-shaped outer segment 114 at the opposite ends thereof on opposite lateral sides of the wearer's neck to secure the collar in place on the wearer with the two segments residing anteriorly of the user's neck.

Like the upper segment of the earlier two-segment collar embodiments, the inner segment 112 receives the wearer's chin on the top side of the inner segment, while the outer segment rests atop the chest area of the wearer and resides sufficient forward of the wearer's neck to prevent any contact of the outer segment with the wearer's chin. The inner segment is connected to the outer segment in a manner enabling relative movement between the two segments. In the illustrated embodiment, this is provided by two lateral connections 116, each spanning outward from a respective lateral side of the inner segment to join to the outer segment near a respective end thereof. The inner segment can move upwardly and downwardly relative to the outer segment in the longitudinal superior-inferior direction of the wearer. The lateral connections 116 may be flexible straps, cords, strings, or other connectors suitable of accommodating motion of the inner segment relative to the outer segment to which they are attached. In a raised position of the inner segment, its topside abuts against the underside of the wearer's chin, and the inner segment may be biased in such an upward direction to encourage the wearer's head into an erected or rearwardly inclined position and encourage the wearer's lower jaw into a closed position, as described above for other collar embodiments.

In FIG. 15A, the inner segment is biased upwardly by a spring 117 attached to the underside of the inner segment and acting against a base 114b that extends inwardly (i.e. posteriorly) toward the wearer's neck atop the chest area beneath the inner segment from the bottom of the outer member 114. Accordingly, the combined base and outer segment may be considered equivalent to the lower segment of the FIG. 1 embodiment. The inner segment is sufficiently tall so that its lower end remains within the void space bound by the outer segment when the inner segment is fully raised in to the jaw-closing position, whereby the inner segment cannot fully escape its proper alignment beneath the chin within this void space.

As an alternative to movement of the entirety of the inner segment relative to the lower segment, as is the case in FIG. 15A, the underside of the inner segment may rest in a stationary position atop the chest area of the wearer or atop a base 114a of the outer segment. FIG. 15C shows such a collar 110′ in which the outer segment 114′ of the collar 110′ lacks a base 114a underlying the inner segment 112′, and instead resides directly atop the wearer's upper chest. In this case, the inner segment 112′ is taller than in FIG. 15A in order to reach the wearer's chin, and use of resilient foam or other resilient compressible material for the inner segment 112′, as shown, and/or incorporation of one or more springs or other resilient members within the internal segment, may be used to allow downward deflection of the topside of the inner segment under lowering of the user's chin with sufficient downforce to compress the inner segment in a manner deflecting its topside downwardly toward its stationary underside. In the collar 110′ of FIGS. 15C and 15D, the connections 116′ between the inner and outer segments needs not necessarily be flexible connections, and could alternatively be rigidly fixed connections at or near the lower end of the inner segment since the jaw-movement is accommodated by compression of the foam or other compressible material of the inner segment, and not by movement of the entire inner segment relative to the outer segment. Accordingly, the lower end of the inner segment need not be movable relative to the outer segment.

Whether the entirety of the inner segment or just the topside thereof can move relative to the outer segment, the topside of the inner segment is movable downwardly with a chin-lowering action (whether forward tilting of the head, or opening of the jaw) by the user. This chin-lowering action is preferably resisted by a spring action of the inner segment, whether this spring action is provided by the particular material structure of the inner segment, or a spring-loaded connection to the outer member, or a combination thereof. Accordingly, as the user's entire head or lower jaw moves downward, the spring type action is sufficient to offer resistance in the opposite direction, thus acting to return the head or lower jaw and return to its initial position (e.g. erected-head, or closed jaw position). The spring force is selected to be sufficient to keep the user's mouth closed when the user's muscles are relaxed during sleep, but low enough that the user can still open their mouth to speak, cough, yawn, consume food or drink, etc. when awake.

The position of the outer segment embracing about three sides of the inner segment in abutment with the wearer's chest, together with the connections between the inner and outer segments stabilize the inner segment (e.g. preventing same from being displaced outwardly from beneath the wearer's chin) while allowing the relative movement between the segments. Thus, the collar has an inner segment with means to support the user's chin on its upper portion and of sufficient height as to contact the users upper chest area with the bottom portion or attach to the outer segment in a manner that allows the inner segment to be stabilized by the outer segment, which contacts the users upper chest area allowing movement of the inner segment including a spring type action. The outer segment is attached to the inner segment in a manner that allows for independent movement of each segment, and that allows the inner segment to move in directions consistent with the movement of the users chin and means of providing a spring type action to return the users chin to its original position while the outer segment provides stability to the overall cervical collar device. While the outer segment of the illustrated embodiment extends not just across the front side of the inner segment, but also rearwardly along the lateral sides of the inner segment, other embodiments may lack such laterally residing portions of the outer segment, in which case the lateral connections 116 that movably support the inner segment may do so by way of connection to the straps, which would thereby indirectly connect the inner segment to the outer segment, instead of having direct connection to the outer segment.

The inner and outer segment structure of the collar 110 may be used with the forgoing supports 22, 22′, 22″ for breathing apparatuses, which may include a hose, nasal pillows, nose masks and other systems consistent with pressurized gas delivery systems. For example, one of the forgoing supports 22, 22′, 22″ may be coupled or affixed to the outer segment of the collar. The nasal breathing apparatus may include means of independent movement such that when the users head moves the breathing apparatus moves independent of the outer segment, returning to its original position by a spring type mechanism.

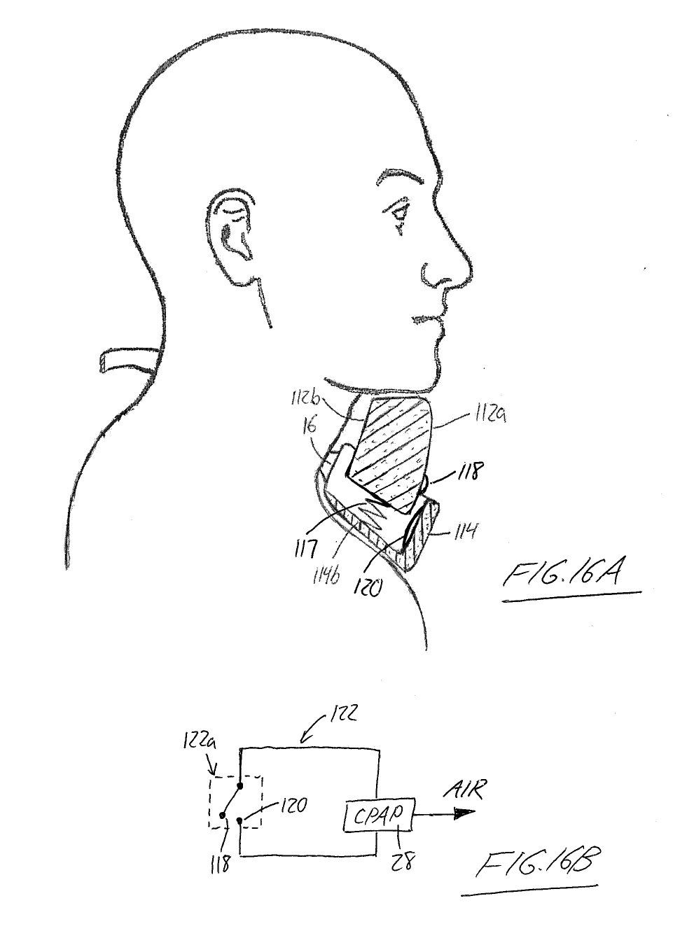

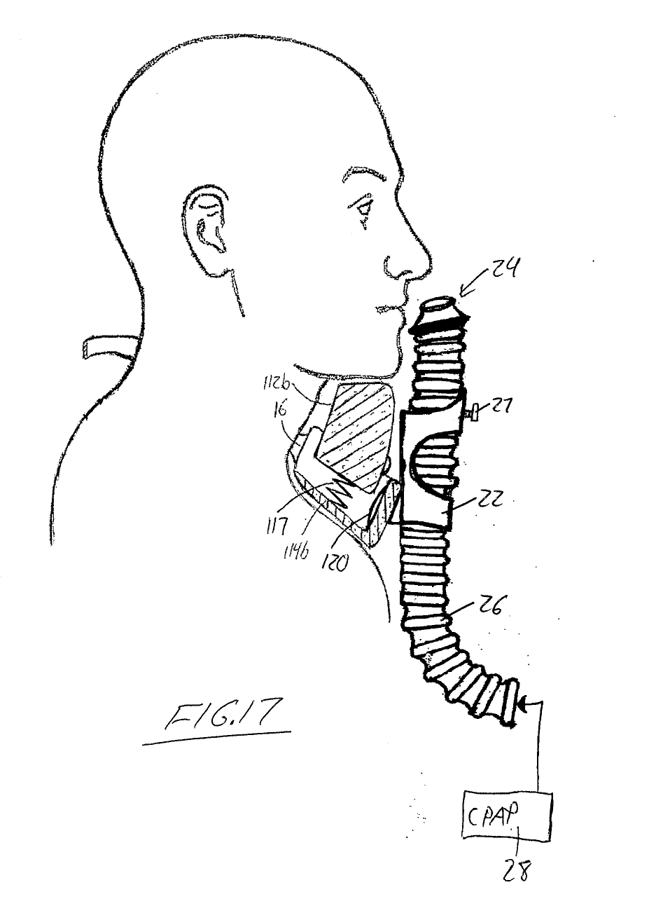

Turning to FIG. 16, cervical collar device 110″ is disclosed that monitors the position of a person's mouth as to an open or closed position. The device is able to detect if the person has their mouth open or closed and may have means to signal various receiving devices, which may include a novel CPAP (continuous positive airway pressure) device.

As CPAP use becomes more widespread and significant are benefits observed, there exist drawbacks to the therapy that are resolved by presently disclosed embodiments. Many CPAP users prefer the nose mask and nasal pillows that allow the mouth to be uncovered, as opposed to full face masks available on the market. The continuous aspect of gas delivery therapy means air pressure is constantly applied to the user in such a manner that if the user opens their mouth to perform common actions including yawning, coughing, speaking or drinking, the air pressure being maintained in the users upper airway and nasal cavities escapes through the users mouth making it very uncomfortable and difficult to yawn, cough or speak and impossible to drink.

The embodiment of FIG. 16 solves the problem by providing a means of detecting when a user's mouth is open and detecting when a user's mouth is closed, and having means to signal a CPAP device that has functionality to receive these signals. The CPAP device, upon receiving a mouth-open signal, immediately stops the air pressure being directed to the user and may include a venting system that discharges air from the CPAP machine and hose system providing instantaneous pressure relief such that the user detects minimal to no air pressure directed outwards of their mouth when it is open. The CPAP device re-starts the supply of pressurized air to the user upon receiving the mouth closed signal.

To accomplish this, FIG. 16A shows a modified version 110″ of the collar of FIG. 15A, which includes electrical contacts 118, 120 on the inner and outer segments 112, 114 such that the opening and closing of the mouth causes the contact means to correspondingly change position in relation to one another. If the users mouth opens, the inner segment moves relative to the outer segment, and so the electrical contact 118 of the inner segment changes position in relation to the outer segment's electrical contact 120, causing an open-mouth signal in a control circuit 122 of the CPAP machine 28, indicating an open-mouth position. If the user's mouth closes the contact means return to the original position indicating a mouth closed position. The illustrated collar 110″ features placement of one electrical contact 118 on the front side 112a of the inner segment 112 at a height thereon that resides in an elevated position outside of the void space of the outer segment 114 when the inner segment 112 is raised into the jaw-closing position, as shown. The other electrical contact 120 on the outer segment 114 spans substantially the full height of the rear side 114a of the outer segment 114 so that the first electrical contact is in contact with the second electrical contact for any open-jaw position, regardless of how far into the void space the inner segment has been forced by the particular degree to which the user's jaw has been opened. Alternatively, a shorter electrical contact at the top end of the outer segment's rear side may be used in combination with a taller electrical contact on the inner segment.

FIG. 16B illustrates how the electrical contacts are wired in a control circuit 122 of a CPAP machine to act as openable/closeable switch 122a, which may be used to switch the output airflow 62 of the CPAP machine 28 on and off in response to closed and open jaw positions, respectively, or to switch the CPAP airflow between high and low pressure states in response to closed and open jaw positions, respectively.

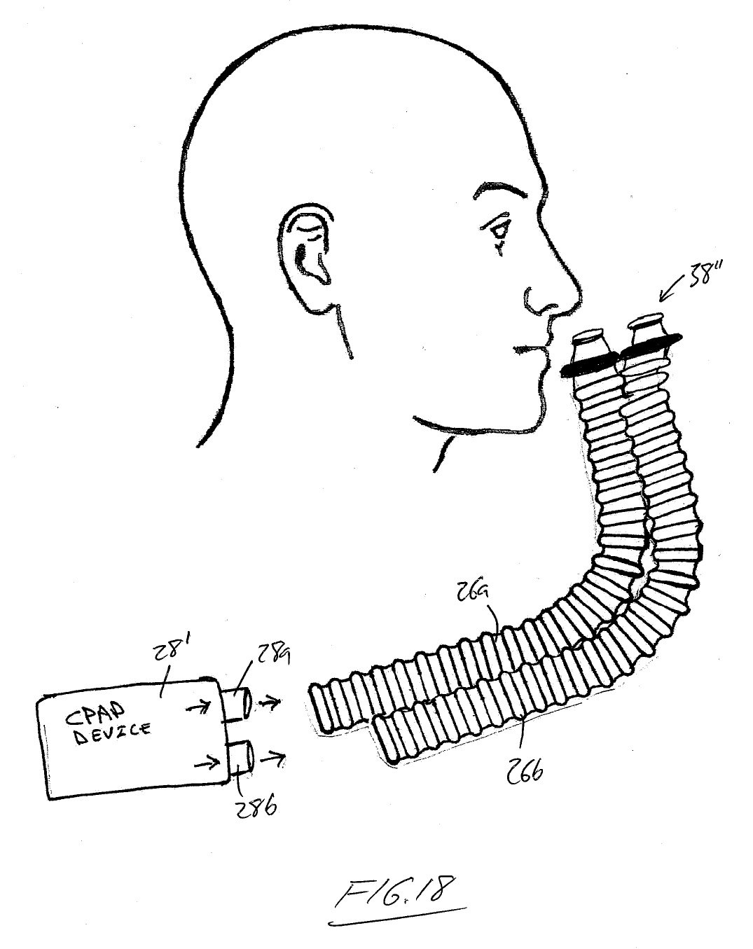

The positional detection means of FIG. 16 may be employed on a cervical collar that also includes any of the above-described means for supporting a patient interface on the cervical collar, as illustrated in FIG. 17 where support 22 is affixed to the front side of the outer segment of the collar. The front side of the outer segment may feature a cut-out or recess therein to receive the support 22 and the hose 26 removably coupled thereto in a position partially recessed or embedded within the collar itself. The placement of the support 22 on the stationary outer segment helps retain the patient interface 24 in place regardless of movement of the inner segment on which the user's chin resides.

Another problem is associated with nasal pillows being used with a CPAP device. CPAP devices in use supply constant pressure to the user's nostrils regardless of the air pathway condition of the individual nasal cavity. Many CPAP users have obstructed airways in the sinus cavities that prevent free airflow. There are various causes of this including sleeping in a side position with the nostril being pressed against the sleeping surface. Many users have considerable pressure applied by the CPAP device such that if one nasal passage is blocked a pressure situation occurs wherein the users nostril can be expanded and even deformed if a continuous blocked situation occurs.

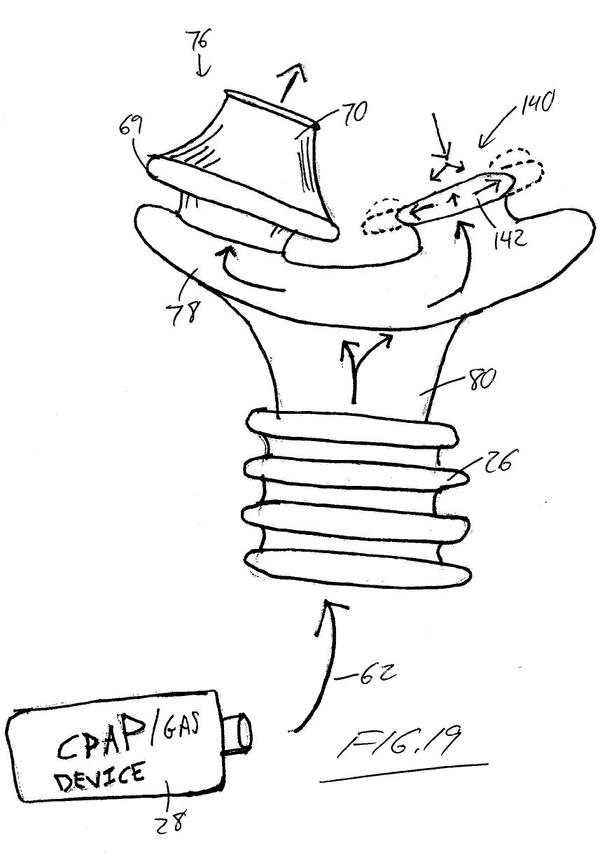

To address this, FIG. 18 illustrates a CPAP device 28′ and hose assembly, including a nasal pillow assembly, which alleviates the described problem. The CPAP nasal pillows 38″ have two independent hose assemblies 26a, 26b connected to two respective air outlets 28a, 28b of the CPCP device 18′ to provide means of accommodating varying pressure to the corresponding nasal passages of the user. The CPAP device has capabilities of monitoring independent nasal cavity air flow, for example using sensors on the nasal pillows 38″, and means of adjusting individual nasal pressure in response to the monitoring such that an obstructed nasal cavity would receive less or no air pressure while an airway obstruction occurs. Basic control logic for varying air pressure supplied to the nasal pillows based on measured flow conditions at the nasal passages is within the ambit of the person of ordinary skill in the art, and therefore not further explained herein.

Another problem associated with nasal pillows is users presently rely on head straps to keep the nasal pillows in place and positioned properly to receive the air pressure supplied by the CPAP device. These straps are uncomfortable and may provide an obstacle to a user decision to continue the CPAP therapy.

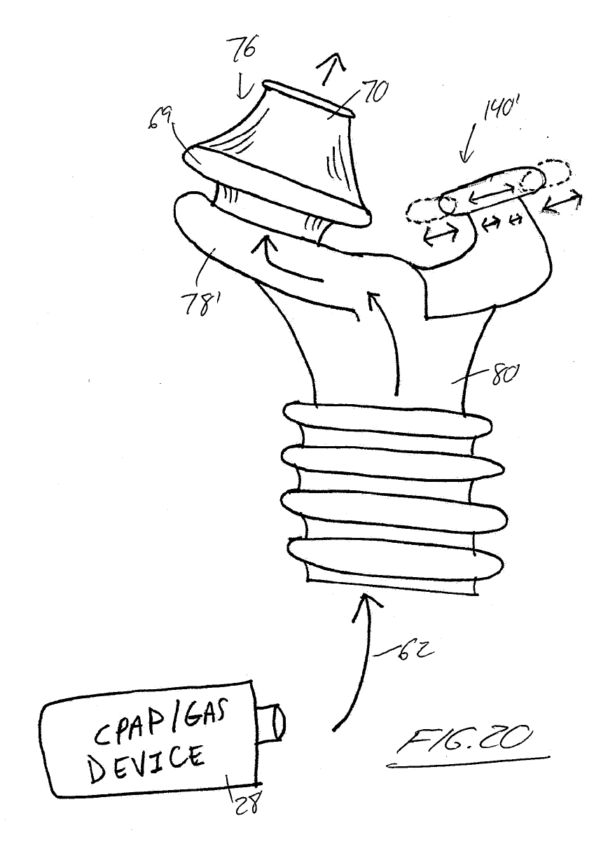

With reference to FIGS. 19 and 20, self-securing nasal pillow assemblies are disclosed that alleviate the need for head straps or other separate fastening systems properly position and secure a nasal pillow assembly.

More particularly, FIG. 19 discloses a CPAP nasal pillow assembly 138 having one modified nasal pillow 140 constructed in a manner that secures the nasal pillow assembly to the user's nose, eliminating the need for head straps or other fastening systems. The one modified nasal pillow 140 is constructed to not allow air to flow through it into the nasal passage, but to instead expand radially within the nasal passage in a manner that allows the nasal pillow assembly to remain in position while air flow is present from the CPAP device. The other nasal pillow 76 is of the type described above with reference to FIG. 13, and thus features a tapered nasal-penetrating body 70 standing upwardly therefrom. A connection channel 78 joins the two nasal pillows 76 together at the underside thereof, and features an inlet port 80 for coupling to the CPAP hose 26 to distribute pressurized air to each nasal pillow through the channel 78. The expandable nasal pillow 140 lacks a nasal-penetrating body 70 with a respective through-flow conduit for delivering air to the respective national passage, and instead of a fixed-size base 69, features a radially expandable chamber 142 in fluid communication with the connection channel 78. With the CPAP device in an inactive state, the nasal-penetrating body 70 of the normal through-flow nasal pillow 76 is inserted into the respective nasal passage, and the unexpanded chamber 142 of the modified nasal pillow 140 is inserted into its respective nasal passage. The CPAP device is switched to an active mode of operation initiating the pressurized air stream 62 to the pillows 76, 140 via the hose 26 and connection channel 78, whereupon pressurization of the connection channel 78 will pneumatically dilate the radially expandable chamber 142, thereby urging the outer periphery thereof into abutment with the walls of the respective nasal passage just above the respective nare. The expanded state of the chamber 142 prevents withdrawal thereof through the nare below, and seals off the nasal passage. This expansion in an outward direction towards the inner sides of the nasal walls prevents back flow air pressure from escaping, and stabilizes the whole nasal pillow assembly to the user's nose. The modified nasal pillow 140 may be designed in a manner to utilize the back flow air pressure 144 caused by the normally operating second nasal pillow 76 to further stabilize the modified nasal pillow 140 in the first nasal passage of the user. The back flow air pressure 144 may be diverted to cause the sideways expansion from the top of the inserted portion.

FIG. 20 shows a similar embodiment that instead of pneumatic operation of the modified pillow 140′ using the CPCP airflow 62 uses a mechanical actuator (not shown) to cause expansion of the modified pillow 140′. In one embodiment, the mechanical actuator may be configured such that the user applies pressure to the bottom part of the modified pillow 140′, the expandable/collapsible top portion 142′ is reduced in size from a default expanded condition and is insertable into the nasal cavity, and when the user releases the pressure from the bottom part, the top portion 142′ expands inside the nasal passage to provide an air tight seal and stabilize the entire nasal pillow assembly to the users nose without the need for head straps. Since air pressure is not used to actuate the expandable pillow, the connection 78′ that joins the two pillows 76, 140′ together only distributes air to the unmodified pillow 76 via an interior flow channel, which is accordingly cut off from the modified pillow 140.

Since various modifications can be made in my invention as herein above described, and many apparently widely different embodiments of same made within the scope of the claims without departure from such scope, it is intended that all matter contained in the accompanying specification shall be interpreted as illustrative only and not in a limiting sense.

1. An apparatus for treatment of sleep apnea or other respiratory disturbances, the apparatus comprising:
a cervical collar comprising a chin-supporting member arranged to be worn anteriorly of the user's neck in a position receiving the user's chin on a top side of the chin-supporting member;
a support feature on the cervical collar at a position residing anteriorly of the user's neck when worn, the support feature being arranged to carry a respiratory patient interface on the cervical collar.

2. The apparatus of claim 1 wherein the cervical collar further comprises a stationary chest-abutting member arranged to be worn anteriorly of the user's neck in a position against the user's chest, and the top side of the chin-supporting member is movable downwardly relative to the chest-abutting member under to accommodate opening of the user's jaw while in a conscious state, the top-side of the chin-supporting member being biased upwardly to force closing of the user's jaw while in an unconscious state, and the support feature being disposed on the stationary chest-abutting member at a position situated anteriorly of the chin-supporting member.

3. The apparatus of claim 1 wherein the support feature is configured to engage an air conveyance conduit to which the respiratory patient interface is coupled to receive supply air from said air conveyance conduit.

4. The apparatus of claim 3 wherein the support feature is arranged to hold the ventilator airway conduit in a position reaching superiorly from the second member to convey air to a nasal respiratory patient interface.

5. The apparatus of claim 4 wherein the support feature comprises a force application member arranged to urge the respiratory patient interface into a working position engaged to a body of the user.

6. The apparatus of claim 5 wherein the respiratory patient interface comprises an air delivery mask and the force application member is arranged to urge the air delivery mask posteriorly of the user for facial engagement with the body of the user.

7. The apparatus of claim 6 wherein the force application member is a spring lever standing upwardly from the support feature at a front end thereof with a distal end of the spring lever biased rearwardly to urge the air delivery mask posteriorly of the user.

8. The apparatus of claim 5 wherein the respiratory patient interface comprises nasal pillows and the force application member is arranged to urge the air nasal pillows superiorly of the user for nasal engagement with the body of the user.

9. The apparatus of claim 8 wherein the support feature comprises a stationary support component held stationary to the second member and a movable support component movably coupled to the stationary support component and biased upwardly relative thereto, the movable support component being arranged to couple to the nasal pillows.

10. The apparatus of claim 9 wherein the movable support component is arranged to couple to the nasal pillows via an air conveyance conduit that is coupled to said nasal pillows to deliver air thereto.

11. The apparatus of claim 1 wherein the support feature comprises an adjustment mechanism for adjusting a position of the respiratory patient interface without disconnection thereof from the support feature.

12. The apparatus of claim 11 wherein the support feature comprises a stationary component coupled to the outer member and a movable component threadingly mated to the stationary component and displaceable relative to the stationary component by relative rotation between the threadingly mated components.

13. The apparatus of claim 12 wherein the threadingly mated components are arranged for relative upward and downward displacement to adjust the position of the respiratory patient interface in a superior-inferior direction of the user.

14. The apparatus of claim 1 wherein the cervical collar comprises an inflatable chamber connected or connectable to a pressurized air supply to enable inflation of the chamber to varying levels to a modify a height at which the support feature is located in order to adjust a position at which the respiratory patient interface is carried.

16. The apparatus of claim 2 wherein the chin-supporting member and the chest-abutting member carry respective electrical contacts thereon that are positioned to come into and out of contact with one another under relative movement between the top side of the chin-supporting member and the chest-abutting member, thereby detecting changes in a positional status of the chin received on the top side of the inner member.

17. The cervical collar of claim 16 wherein the respective electrical contacts are positioned to come into and out of contact with one another under relative movement between the top side of the inner member and the chest-abutting member in a superior-anterior direction of the user, thereby detecting changes in an open/closed state of the user's jaw.

18. The cervical collar of claim 17 wherein the electrical contacts are positioned to come into contact under inferior urging of the top side of the chin-supporting member toward the chest-abutting member by a jaw-opening action of the user.

19. The cervical collar of claim 16 wherein the contacts are connected to a control circuit of a respiratory ventilation machine to alter an output thereof in response to a change in a state of contact between the electric contacts.

20. An apparatus for treatment of sleep apnea or other respiratory disturbances, the apparatus comprising:
a cervical collar comprising:
a stationary chest-abutting member arranged to be worn anteriorly of the user's neck in a position against the user's chest,
a chin-supporting member arranged to be worn anteriorly of the user's neck in a position receiving the user's chin on a top side of the chin-supporting member, the top side of the chin-supporting member being movable downwardly relative to the chest-abutting member under to accommodate opening of the user's jaw while in a conscious state, and being biased upwardly to force closing of the user's jaw while in an unconscious state; and
a support feature on the stationary chest-abutting member arranged to carry a respiratory patient interface thereon.
